SEQUENCE LISTING (34 sequences)

SEQ ID NO: 1 - protein, 7 aa, Human immunodeficiency virus
KRRQRRR

SEQ ID NO: 2 - protein, 16 aa, Homo sapiens
RQIKIWFQNRRMKWKK

SEQ ID NO: 3 - protein, 34 aa, Human herpesvirus 1
DAATATRGRSAASRPTERPRAPARSASRPRRPVD

SEQ ID NO: 4 - protein, 16 aa, Homo sapiens
AAVALLPAVLLALLAP

SEQ ID NO: 5 - protein, 16 aa, Homo sapiens
AAVLLPVLLPVLLAAP

SEQ ID NO: 6 - protein, 15 aa, Homo sapiens
VTVLALGALAGVGVG

SEQ ID NO: 7 - protein, 17 aa, Homo sapiens
GALFLGWLGAAGSTMGA

SEQ ID NO: 8 - protein, 17 aa, Caiman crocodylus
MGLGLHLLVLAAALQGA

SEQ ID NO: 9 - protein, 24 aa, Homo sapiens
LGTYTQDFNKFHTFPQTAIGVGAP

SEQ ID NO: 10 - protein, 26 aa, Artificial Sequence
  Description of Artificial Sequence Synthetic peptide
GWTLNSAGYLLKINLKALAALAKKIL

SEQ ID NO: 11 - protein, 16 aa, Artificial Sequence
  Description of Artificial Sequence Synthetic peptide
TPPKKKRKVEDPKKKK

SEQ ID NO: 12 - protein, 7 aa, Artificial Sequence
  Description of Artificial Sequence Synthetic peptide
RRRRRRR

SEQ ID NO: 13 - protein, 18 aa, Artificial Sequence
  Description of Artificial Sequence Synthetic peptide
KLALKLALKALKAALKLA

SEQ ID NO: 14 - protein, 16 aa, Influenza A virus
GLFGAIAGFIENGWEG

SEQ ID NO: 15 - protein, 16 aa, Sendai virus
FFGAVIGTIALGVATA

SEQ ID NO: 16 - protein, 16 aa, Respiratory syncytial virus
FLGFLLGVGSAIASGV

SEQ ID NO: 17 - protein, 16 aa, Human immunodeficiency virus
GVFVLGFLGFLATAGS

SEQ ID NO: 18 - protein, 16 aa, Ebola virus
GAAIGLAWIPYFGPAA

SEQ ID NO: 19 - protein, 56 aa, Homo sapiens
ACTCPYCKDSEGRGSGDPGKKKQHICHIQGCGKVYGKTSHLRAHLRWHTGERPFMC

SEQ ID NO: 20 - protein, 54 aa, Homo sapiens
ACTCPNCKDGEKRSGEQGKKKHVCHIPDCGKTRFKTSLLRAHVRLHTGERPFVC

SEQ ID NO: 21 - protein, 55 aa, Homo sapiens
ACTCPNCKEGGGRGTNLGKKKQHICHIPGCGKVYGKTSHLRAHLRWHSGERPFVC

SEQ ID NO: 22 - protein, 56 aa, Homo sapiens
ACSCPNCREGEGRGSNEPGKKKQHICHIEGCGKVYGKTSHLRAHLRWHTGERPFIC

SEQ ID NO: 23 - protein, 60 aa, Drosophila melanogaster
RCTCPNCTNEMSGLPPIVGPDERGRKQHICHIPGCERLYGKASHLKTHLRWHTGERPFLC

SEQ ID NO: 24 - protein, 58 aa, Drosophila melanogaster
TCDCPNCQEAERLGPAGVHLRKKNIHSCHIPGCGKVYGKTSHLKAHLRWHTGERPFVC

SEQ ID NO: 25 - protein, 53 aa, Caenorhabditis elegans
RCTCPNCKAIKHGDRGSQHTHLCSVPGCGKTYKKTSHLRAHLRKHTGDRPFVC

SEQ ID NO: 26 - protein, 56 aa, Caenorhabditis elegans
PQISLKKKIFFFIFSNFRGDGKSRIHICHLCNKTYGKTSHLRAHLRGHAGNKPFAC

SEQ ID NO: 27 - protein, 31 aa, Artificial Sequence
  Description of Artificial Sequence Synthetic peptide
WWETWKPFQCRICMRNFSTRQARRNHRRRHR

SEQ ID NO: 28 - protein, 16 aa, Artificial Sequence
  Description of Artificial Sequence Synthetic peptide
GKINLKALAALAKKIL

SEQ ID NO: 29 - protein, 16 aa, Artificial Sequence
  Description of Artificial Sequence Synthetic peptide
RVIRVWFQNKRCKDKK

SEQ ID NO: 30 - protein, 39 aa, Artificial Sequence
  Description of Artificial Sequence Synthetic peptide
GRKKRRQRRRPPQGRKKRRQRRRPPQGRKKRRQRRRPPQ

SEQ ID NO: 31 - protein, 22 aa, Artificial Sequence
  Description of Artificial Sequence Synthetic peptide
GEQIAQLIAGYIDIILKKKKSK

SEQ ID NO: 32 - protein, 18 aa, Artificial Sequence
  Description of Artificial Sequence Synthetic peptide
CNGRCGGKKKLKLLLKLL

SEQ ID NO: 33 - protein, 20 aa, Artificial Sequence
  Description of Artificial Sequence Synthetic peptide
LRKLRKRLLRLRKLRKRLLR

SEQ ID NO: 34 - protein, 23 aa, Artificial Sequence
  Description of Artificial Sequence Synthetic peptide
  MOD_RES (2)..(5): Variable amino acid and this region may encompass 2 or 4 residues
  MOD_RES (7)..(18): Variable amino acid
  MOD_RES (20)..(22): Variable amino acid
CXXXXCXXXXXXXXXXXXHXXXH

(19) United States
(12) Patent Application Publication
(10) Pub. No.: US 2007/0154449 A1
(43) Pub. Date: Jul. 5, 2007

(54) TIGHT JUNCTION MODULATING PEPTIDES FOR ENHANCED MUCOSAL DELIVERY OF THERAPEUTIC COMPOUNDS

(75) Inventors: Kunyuan Cui, Bothell, WA (US); Shu-Chih Chen Quay, Seattle, WA (US); Kristine T. Fry, Seattle, WA (US)

Correspondence Address: NASTECH PHARMACEUTICAL COMPANY INC, 3830 MONTE VILLA PARKWAY, BOTHELL, WA, 98021-7266, US

(73) Assignee: Nastech Pharmaceutical Company Inc.

(21) Appl. No.: 11/611,289

(22) Filed: Dec. 15, 2006

Related U.S. Application Data
(60) Provisional application No. 60/750,886, filed on Dec. 16, 2005.

(51) Int. Cl. A61K 38/28; A61K 38/21; A61K 38/19; A61K 38/10; A61K 38/18; A61K 9/48

(52) U.S. Cl. 424/85.1; 424/451; 424/85.4; 514/15; 530/326; 514/3; 514/12

(57) ABSTRACT

Compositions and methods are provided that include a biologically active agent and a permeabilizing agent effective to enhance mucosal delivery of the biologically active agent in a mammalian subject, in which the permeabilizing peptide is a tight junction modulating peptide (TJMP), a TJMP analogue, a conjugate of a TJMP, a conjugate of a TJMP analogue, or complexes thereof. The permeabilizing agent reversibly enhances mucosal epithelial paracellular transport, typically by modulating epithelial junctional structure and/or physiology at a mucosal epithelial surface in the subject.

This application claims the benefit under 35 U.S.C. § 119(e) of U.S. Provisional Application No. 60/750,886, filed Dec. 16, 2005, which is hereby incorporated by reference in its entirety.

### BACKGROUND OF THE INVENTION

A fundamental concern in the treatment of any disease or condition is ensuring the safe and effective delivery of a therapeutic agent drug to the patient. Traditional routes of delivery for therapeutic agents include intravenous injection and oral administration. However, these delivery methods suffer from several disadvantages and thus alternative delivery systems are needed to overcome these shortcomings.

A major disadvantage of drug administration by injection is that trained personnel are often required to administer the drug. Additionally, trained personal are put in harms way when administering a drug by injection. For self-administered drugs, many patients are reluctant or unable to give themselves injections on a regular basis. Injection is also associated with increased risks of infection. Other disadvantages of drug injection include variability of delivery results between individuals, as well as unpredictable intensity and duration of drug action.

The oral administration of certain therapeutic agents exhibit very low bioavailability and considerable time delay in action when given by this route due to hepatic first-pass metabolism and degradation in the gastrointestinal tract.

Mucosal administration of therapeutic compounds offers certain advantages over injection and other modes of administration, for example in terms of convenience and speed of delivery, as well as by reducing or eliminating compliance problems and side effects that attend delivery. However, mucosal delivery of biologically active agents is limited by mucosal barrier functions and other factors. Epithelial cells make up this mucosal barrier and provide a crucial interface between the external environment and mucosal and submucosal tissues and extracellular compartments. One of the most important functions of mucosal epithelial cells is to determine and regulate mucosal permeability. In this context, epithelial cells create selective permeability barriers between different physiological compartments. Selective permeability is the result of regulated transport of molecules through the cytoplasm (the transcellular pathway) and the regulated permeability of the spaces between the cells (the paracellular pathway).

Intercellular junctions between epithelial cells are known to be involved in both the maintenance and regulation of the epithelial barrier function, and cell-cell adhesion. Tight junctions (TJ) of epithelial and endothelial cells are particularly important for cell-cell junctions that regulate permeability of the paracellular pathway, and also divide the cell surface into apical and basolateral compartments. Tight junctions form continuous circumferential intercellular contacts between epithelial cells and create a regulated barrier to the paracellular movement of water, solutes, and immune cells. They also provide a second type of barrier that contributes to cell polarity by limiting exchange of membrane lipids between the apical and basolateral membrane domains.

In the context of drug delivery, the ability of drugs to permeate epithelial cell layers of mucosal surfaces, unassisted by delivery-enhancing agents, appears to be related to a number of factors, including molecular size, lipid solubility, and ionization. In general, small molecules, less than about 300-1,000 daltons, are often capable of penetrating mucosal barriers, however, as molecular size increases, permeability decreases rapidly. For these reasons, mucosal drug administration typically requires larger amounts of drug than administration by injection. Other therapeutic compounds, including large molecule drugs, are often refractory to mucosal delivery. In addition to poor intrinsic permeability, large macromolecular drugs are often subject to limited diffusion, as well as lumenal and cellular enzymatic degradation and rapid clearance at mucosal sites. Thus, in order to deliver these larger molecules in therapeutically effective amounts, cell permeation enhancing agents are required to aid their passage across these mucosal surfaces and into systemic circulation where they may quickly act on the target tissue.

The current work explores the therapeutic utility of novel tight junction modulating peptides (TMJP) for drug delivery across a mucosal surface, for example intranasal (IN) drug delivery. Both in vitro and in vivo assessments suggest that the TJMPs represent a promising new approach for improving the delivery of small molecules and macromolecules across mucosal surfaces.

### DETAILED DESCRIPTION OF INVENTION

The instant invention satisfies the foregoing needs and fulfills additional objects and advantages by providing novel pharmaceutical compositions that include the novel use of newly discovered tight junction-opening peptides to enhance mucosal delivery of the biologically active agent in a mammalian subject. The tight junction-opening peptides that can be used to open the tight junctions in mucosal tissue, especially nasal tissue, include:

| | |
|---|---|
| CNGRCGGKKKLKLLLKLL | (SEQ ID NO: 32) |
| LRKLRKRLLRLRKLRKRLLR-amide | (SEQ ID NO: 33) |

The permeabilizing agent reversibly enhances mucosal epithelial paracellular transport, typically by modulating epithelial tight junction structures and/or physiology at a mucosal epithelial surface in the subject. This effect typically involves inhibition by the permeabilizing agent of homotypic or heterotypic binding between epithelial membrane adhesive proteins of neighboring epithelial cells. Target proteins for this blockade of homotypic or heterotypic binding can be selected from various related junctional adhesion molecules (JAMs), occluding, or claudins.

Epithelial Cell Biology

Epithelial cells provide a crucial interface between the external environment and mucosal and submucosal tissues and extracellular compartments. One of the most important functions of mucosal epithelial cells is to determine and regulate mucosal permeability. In this context, epithelial cells create selective permeability barriers between different physiological compartments. Selective permeability is the result of regulated transport of molecules through the cytoplasm (the transcellular pathway) and the regulated permeability of the spaces between the cells (the paracellular pathway).

Intercellular junctions between epithelial cells are known to be involved in both the maintenance and regulation of the epithelial barrier function, and cell-cell adhesion. The tight junction (TJ) of epithelial and endothelial cells is a particularly important cell-cell junction that regulates permeability of the paracellular pathway, and also divides the cell surface into apical and basolateral compartments. Tight junctions form continuous circumferential intercellular contacts between epithelial cells and create a regulated barrier to the paracellular movement of water, solutes, and immune cells. They also provide a second type of barrier that contributes to cell polarity by limiting exchange of membrane lipids between the apical and basolateral membrane domains.

Tight junctions are thought to be directly involved in barrier and fence functions of epithelial cells by creating an intercellular seal to generate a primary barrier against the diffusion of solutes through the paracellular pathway, and by acting as a boundary between the apical and basolateral plasma membrane domains to create and maintain cell polarity, respectively. Tight junctions are also implicated in the transmigration of leukocytes to reach inflammatory sites. In response to chemoattractants, leukocytes emigrate from the blood by crossing the endothelium and, in the case of mucosal infections, cross the inflamed epithelium. Transmigration occurs primarily along the paracellular rout and appears to be regulated via opening and closing of tight junctions in a highly coordinated and reversible manner.

Numerous proteins have been identified in association with TJs, including both integral and peripheral plasma membrane proteins. Current understanding of the complex structure and interactive functions of these proteins remains limited. Among the many proteins associated with epithelial junctions, several categories of trans-epithelial membrane proteins have been identified that may function in the physiological regulation of epithelial junctions. These include a number of “junctional adhesion molecules” (JAMs) and other TJ-associated molecules designated as occluding, claudins, and zonulin.

JAMs, occludin, and claudin extend into the paracellular space, and these proteins in particular have been contemplated as candidates for creating an epithelial barrier between adjacent epithelial cells and regulatable channels through epithelial cell layers. In one model, occludin, claudin, and JAM have been proposed to interact as homophilic binding partners to create a regulated barrier to paracellular movement of water, solutes, and immune cells between epithelial cells.

A cDNA encoding murine junctional adhesion molecule-1 (JAM-1) has been cloned and corresponds to a predicted type I transmembrane protein (comprising a single transmembrane domain) with a molecular weight of approximately 32-kD (Williams, et al., Molecular Immunology 36:1175-1188, 1999; Gupta, et al., IUBMB Life 50:51-56, 2000; Ozaki, et al., J. Immunol. 163:553-557, 1999; Martin-Padura, et al., J. Cell Biol. 142:117-127, 1998). The extracellular segment of the molecule comprises two Ig-like domains described as an amino terminal “VH-type” and a carboxy-terminal “C2-type” carboxy-terminal β-sandwich fold (Bazzoni et al., Microcirculation 8:143-152, 2001). Murine JAM-1 also contains two sites for N-glycosylation, and a cytoplasmic domain. The JAM-1 protein is a member of the immunoglobulin (Ig) superfamily and localizes to tight junctions of both epithelial and endothelial cells. Ultrastructural studies indicate that JAM-1 is limited to the membrane regions containing fibrils of occludin and claudin.

Another JAM family member, designated “Vascular endothelial junction-associated molecule” (VE-JAM), contains two extracellular immunoglobulin-like domains, a transmembrane domain, and a relatively short cytoplasmic tail. VE-JAM is principally localized to intercellular boundaries of endothelial cells (Palmeri, et al., J. Biol. Chem. 275:19139-19145, 2000). VE-JAM is highly expressed highly by endothelial cells of venules, and is also expressed by endothelia of other vessels. Another reported JAM family member, JAM-3, has a predicted amino acid sequence that displays 36% and 32% identity, respectively, to JAM-2 and JAM-1. JAM-3 shows widespread tissue expression with higher levels apparent in the kidney, brain, and placenta. At the cellular level, JAM-3 transcript is expressed within endothelial cells. JAM-3 and JAM-2 have been reported to be binding partners. In particular, the JAM-3 ectodomain reportedly binds to JAM2-Fc. JAM-3 protein is up-regulated on peripheral blood lymphocytes following activation. (Pia Arrate, et al., J. Biol. Chem. 276: 45826-45832, 2001).

Another proposed trans-membrane adhesive protein involved in epithelial tight junction regulation is occludin. Occludin is an approximately 65-kD type II transmembrane protein composed of four transmembrane domains, two extracellular loops, and a large C-terminal cytosolic domain [Furuse, et al., J. Cell Biol. 123:1777-1788, 1993; Furuse, et al., J. Cell Biol. 127:1617-1626, 1994]. When observed by immuno-freeze fracture electron microscopy, occludin is concentrated directly within the tight junction fibrils (Fujimoto, J. Cell Sci. 108:3443-3449, 1995).

Two additional integral membrane proteins of the tight junction, claudin-1 and claudin-2, were identified by direct biochemical fractionation of junction-enriched membranes from chicken liver [Furuse, et al., J. Cell Biol. 141:1539-1550, 1998]. Claudin-1 and claudin-2 were found to copurify with occludin as a broad approximately 22-kD gel band on sodium dodecyl sulfate-polyacrylamide gel electrophoresis. The deduced sequences of two closely related proteins cloned from a mouse cDNA library predict four transmembrane helices, two short extracellular loops, and short cytoplasmic N- and C-termini. Despite topologies similar to that of occludin, they share no sequence homology. Subsequently, six more claudin gene products (claudin-3 through claudin-8) have been cloned and have been shown to localize within tight junction fibrils, as determined by immunogold freeze fracture labeling [Morita, et al., Proc. Natl. Acad. Sci. USA 96:511-516, 1999]. Given that a barrier remains in the absence of occludin, claudin-1 through claudin-8 have been considered as candidates for the primary seal-forming elements of the extracellular space.

Other cytoplasmic proteins that have been localized to epithelial junctions include zonulin, symplekin, cingulin, and 7H6. Zonulins reportedly are cytoplasmic proteins that bind the cytoplasmic tail of occludin. Representing this family of proteins are “ZO-1, ZO-2, and ZO-3.” Zonulin is postulated to be a human protein analogue of the Vibrio cholerae derived zonula occludens toxin (ZOT).

Zonulin likely plays a role in tight junction regulation during developmental, physiological, and pathological processes—including tissue morphogenesis, movement of fluid, macromolecules and leukocytes between the intestinal lumen and the interstitium, and inflammatory/autoimmune disorders [see, e.g., Wang, et al., J. Cell Sci. 113:4435-40, 2000; Fasano, et al., Lancet 355:1518-9, 2000; Fasano, Ann. N.Y. Acad. Sci. 915:214-222, 2000]. Zonulin expression increased in intestinal tissues during the acute phase of coeliac disease, a clinical condition in which tight junctions are opened and permeability is increased. Zonulin induces tight junction disassembly and a subsequent increase in intestinal permeability in non-human primate intestinal epithelia in vitro.

Comparison of amino acids in the active V. cholerae ZOT fragment and human zonulin identified a putative receptor binding domain within the N-terminal region of the two proteins. The ZOT biologically active domain increases intestinal permeability by interacting with a mammalian cell receptor with subsequent activation of intracellular signaling leading to the disassembly of the intercellular tight junction. The ZOT biologically active domain has been localized toward the carboxyl terminus of the protein and coincides with the predicted cleavage product generated by V. cholerae. This domain shares a putative receptor-binding motif with zonulin, the ZOT mammalian analogue. Amino acid comparison between the ZOT active fragment and zonulin, combined with site-directed mutagenesis experiments, suggest an octapeptide receptor-binding domain toward the amino terminus of processed ZOT and the amino terminus of zonulin. [Di Pierro, et al., J. Biol. Chem. 276:19160-19165, 2001]. ZO-1 reportedly binds actin, AF-6, ZO-associated kinase (ZAK), fodrin, and [symbol] -catenin.

Permeabilizing peptides for use within the invention include natural or synthetic, therapeutically or prophylactically active, peptides (comprised of two or more covalently linked amino acids), proteins, peptide or protein fragments, peptide or protein analogs, peptide or protein mimetics, and chemically modified derivatives or salts of active peptides or proteins. Thus, as used herein, the term “permeabilizing peptide” will often be intended to embrace all of these active species, i.e., peptides and proteins, peptide and protein fragments, peptide and protein analogs, peptide and protein mimetics, and chemically modified derivatives and salts of active peptides or proteins. Often, the permeabilizing peptides or proteins are muteins that are readily obtainable by partial substitution, addition, or deletion of amino acids within a naturally occurring or native (e.g., wild-type, naturally occurring mutant, or allelic variant) peptide or protein sequence. Additionally, biologically active fragments of native peptides or proteins are included. Such mutant derivatives and fragments substantially retain the desired biological activity of the native peptide or proteins. In the case of peptides or proteins having carbohydrate chains, biologically active variants marked by alterations in these carbohydrate species are also included within the invention.

The permeabilizing peptides, proteins, analogs and mimetics for use within the methods and compositions of the invention are often formulated in a pharmaceutical composition comprising a mucosal delivery-enhancing or permeabilizing effective amount of the permeabilizing peptide, protein, analog or mimetic that reversibly enhances mucosal epithelial paracellular transport by modulating epithelial junctional structure and/or physiology in a mammalian subject.

Biologically Active Agents

The methods and compositions of the present invention are directed toward enhancing mucosal, e.g., intranasal, delivery of a broad spectrum of biologically active agents to achieve therapeutic, prophylactic or other desired physiological results in mammalian subjects. As used herein, the term “biologically active agent” encompasses any substance that produces a physiological response when mucosally administered to a mammalian subject according to the methods and compositions herein. Useful biologically active agents in this context include therapeutic or prophylactic agents applied in all major fields of clinical medicine, as well as nutrients, cofactors, enzymes (endogenous or foreign), antioxidants, and the like. Thus, the biologically active agent may be water-soluble or water-insoluble, and may include higher molecular weight proteins, peptides, carbohydrates, glycoproteins, lipids, and/or glycolipids, nucleosides, polynucleotides, and other active agents.

Useful pharmaceutical agents within the methods and compositions of the invention include drugs and macromolecular therapeutic or prophylactic agents embracing a wide spectrum of compounds, including small molecule drugs, peptides, proteins, and vaccine agents. Exemplary pharmaceutical agents for use within the invention are biologically active for treatment or prophylaxis of a selected disease or condition in the subject. Biological activity in this context can be determined as any significant (i.e., measurable, statistically significant) effect on a physiological parameter, marker, or clinical symptom associated with a subject disease or condition, as evaluated by an appropriate in vitro or in vivo assay system involving actual patients, cell cultures, sample assays, or acceptable animal models.

The methods and compositions of the invention provide unexpected advantages for treatment of diseases and other conditions in mammalian subjects, which advantages are mediated, for example, by providing enhanced speed, duration, fidelity or control of mucosal delivery of therapeutic and prophylactic compounds to reach selected physiological compartments in the subject (e.g., into or across the nasal mucosa, into the systemic circulation or central nervous system (CNS), or to any selected target organ, tissue, fluid or cellular or extracellular compartment within the subject).

In various exemplary embodiments, the methods and compositions of the invention may incorporate one or more biologically active agent(s) selected from:

opiods or opiod antagonists, such as morphine, hydromorphone, oxymorphone, lovorphanol, levallorphan, codeine, nalmefene, nalorphine, nalozone, naltrexone, buprenorphine, butorphanol, and nalbufine;

corticosterones, such as cortisone, hydrocortisone, fludrocortisone, prednisone, prednisolone, methylprednisolone, triamcinolone, dexamethoasone, betamethoasone, paramethosone, and fluocinolone;

other anti-inflammatories, such as colchicine, ibuprofen, indomethacin, and piroxicam; anti-viral agents such as acyclovir, ribavarin, trifluorothyridine, Ara-A (Arabinofuranosyladenine), acylguanosine, nordeoxyguanosine, azidothymidine, dideoxyadenosine, and dideoxycytidine; antiandrogens such as spironolactone;

androgens, such as testosterone;

estrogens, such as estradiol;

progestins;

muscle relaxants, such as papaverine;

vasodilators, such as nitroglycerin, vasoactive intestinal peptide and calcitonin related gene peptide;

antihistamines, such as cyproheptadine;

agents with histamine receptor site blocking activity, such as doxepin, imipramine, and cimetidine;

antitussives, such as dextromethorphan; neuroleptics such as clozaril; antiarrhythmics;

antiepileptics;

enzymes, such as superoxide dismutase and neuroenkephalinase;

anti-fungal agents, such as amphotericin B, griseofulvin, miconazole, ketoconazole, tioconazol, itraconazole, and fluconazole;

antibacterials, such as penicillins, cephalosporins, tetracyclines, aminoglucosides, erythromicin, gentamicins, polymyxin B;

anti-cancer agents, such as 5-fluorouracil, bleomycin, methotrexate, and hydroxyurea, dideoxyinosine, floxuridine, 6-mercaptopurine, doxorubicin, daunorubicin, I-darubicin, taxol and paclitaxel (optionally provided in a bimodal emulsion, e.g., as described in U.S. patent application Ser. No. 09/631,246, filed by Quay on Aug. 2, 2000);

antioxidants, such as tocopherols, retinoids, carotenoids, ubiquinones, metal chelators, and phytic acid;

antiarrhythmic agents, such as quinidine; and

antihypertensive agents such as prazosin, verapamil, nifedipine, and diltiazem; analgesics such as acetaminophen and aspirin;

monoclonal and polyclonal antibodies, including humanized antibodies, and antibody fragments;

anti-sense oligonucleotides; and

RNA, DNA and viral vectors comprising genes encoding therapeutic peptides and proteins.

In addition to these exemplary classes and species of active agents, the methods and compositions of the invention embrace any physiologically active agent, as well as any combination of multiple active agents, described above or elsewhere herein or otherwise known in the art, that is individually or combinatorially effective within the methods and compositions of the invention for treatment or prevention of a selected disease or condition in a mammalian subject (see, Physicians' Desk Reference, published by Medical Economics Company, a division of Litton Industries, Inc).

Regardless of the class of compound employed, the biologically active agent for use within the invention will be present in the compositions and methods of the invention in an amount sufficient to provide the desired physiological effect with no significant, unacceptable toxicity or other adverse side effects to the subject. The appropriate dosage levels of all biologically active agents will be readily determined without undue experimentation by the skilled artisan. Because the methods and compositions of the invention provide for enhanced delivery of the biologically active agent(s), dosage levels significantly lower than conventional dosage levels may be used with success. In general, the active substance will be present in the composition in an amount of from about 0.01% to about 50%, often between about 0.1% to about 20%, and commonly between about 1.0% to 5% or 10% by weight of the total intranasal formulation depending upon the particular substance employed.

As used herein, the terms biolotically active “peptide” and “protein” include polypeptides of various sizes, and do not limit the invention to amino acid polymers of any particular size. Peptides from as small as a few amino acids in length, to proteins of any size, as well as peptide-peptide, protein-protein fusions and protein-peptide fusions, are encompassed by the present invention, so long as the protein or peptide is biologically active in the context of eliciting a specific physiological, immunological, therapeutic, or prophylactic effect or response.

The instant invention provides novel formulations and coordinate administration methods for enhanced mucosal delivery of biologically active peptides and proteins. Illustrative examples of therapeutic peptides and proteins for use within the invention include, but are not limited to: tissue plasminogen activator (TPA), epidermal growth factor (EGF), fibroblast growth factor (FGF-acidic or basic), platelet derived growth factor (PDGF), transforming growth factor (TGF-alpha or beta), vasoactive intestinal peptide, tumor necrosis factor (TNF), hypothalmic releasing factors, prolactin, thyroid stimulating hormone (TSH), adrenocorticotropic hormone (ACTH), parathyroid hormone (PTH), follicle stimulating hormone (FSF), luteinizing hormone releasing hormone (LHRH), endorphins, glucagon, calcitonin, oxytocin, carbetocin, aldoetecone, enkaphalins, somatostin, somatotropin, somatomedin, gonadotrophin, estrogen, progesterone, testosterone, alpha-melanocyte stimulating hormone, non-naturally occurring opiods, lidocaine, ketoprofen, sufentainil, terbutaline, droperidol, scopolamine, gonadorelin, ciclopirox, olamine, buspirone, calcitonin, cromolyn sodium or midazolam, cyclosporin, lisinopril, captopril, delapril, cimetidine, ranitidine, famotidine, superoxide dismutase, asparaginase, arginase, arginine deaminease, adenosine deaminase ribonuclease, trypsin, chemotrypsin, and papain. Additional examples of useful peptides include, but are not limited to, bombesin, substance P, vasopressin, alpha-globulins, transferrin, fibrinogen, beta-lipoproteins, beta-globulins, prothrombin, ceruloplasmin, alpha₂-glycoproteins, alpha₂-globulins, fetuin, alpha₁-lipoproteins, alpha₁-globulins, albumin, prealbumin, and other bioactive proteins and recombinant protein products.

In more detailed aspects of the invention, methods and compositions are provided for enhanced mucosal delivery of specific, biologically active peptide or protein therapeutics to treat (i.e., to eliminate, or reduce the occurrence or severity of symptoms of) an existing disease or condition, or to prevent onset of a disease or condition in a subject identified to be at risk for the subject disease or condition. Biologically active peptides and proteins that are useful within these aspects of the invention include, but are not limited to hematopoietics; antiinfective agents; antidementia agents; antiviral agents; antitumoral agents; antipyretics; analgesics; antiinflammatory agents; antiulcer agents; antiallergic agents; antidepressants; psychotropic agents; cardiotonics; antiarrythmic agents; vasodilators; antihypertensive agents such as hypotensive diuretics; antidiabetic agents; anticoagulants; cholesterol lowering agents; therapeutic agents for osteoporosis; hormones; antibiotics; vaccines; and the like.

Biologically active peptides and proteins for use within these aspects of the invention include, but are not limited to, cytokines; peptide hormones; growth factors; factors acting on the cardiovascular system; cell adhesion factors; factors acting on the central and peripheral nervous systems; factors acting on humoral electrolytes and hemal organic substances; factors acting on bone and skeleton growth or physiology; factors acting on the gastrointestinal system; factors acting on the kidney and urinary organs; factors acting on the connective tissue and skin; factors acting on the sense organs; factors acting on the immune system; factors acting on the respiratory system; factors acting on the genital organs; and various enzymes.

For example, hormones which may be administered within the methods and compositions of the present invention include androgens, estrogens, prostaglandins, somatotropins, gonadotropins, interleukins, steroids and cytokines.

Vaccines which may be administered within the methods and compositions of the present invention include bacterial and viral vaccines, such as vaccines for hepatitis, influenza, respiratory syncytial virus (RSV), parainfluenza virus (PIV), tuberculosis, canary pox, chicken pox, measles, mumps, rubella, pneumonia, and human immunodeficiency virus (HIV).

Bacterial toxoids which may be administered within the methods and compositions of the present invention include diphtheria, tetanus, pseudonomas and mycobactrium tuberculosis.

Examples of specific cardiovascular or thromobolytic agents for use within the invention include hirugen, hirulos and hirudine.

Antibody reagents that are usefully administered with the present invention include monoclonal antibodies, polyclonal antibodies, humanized antibodies, antibody fragments, fusions and multimers, and immunoglobins.

As used herein, the term “conservative amino acid substitution” refers to the general interchangeability of amino acid residues having similar side chains. For example, a commonly interchangeable group of amino acids having aliphatic side chains is alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Examples of conservative substitutions include the substitution of a non-polar (hydrophobic) residue such as isoleucine, valine, leucine or methionine for another. Likewise, the present invention contemplates the substitution of a polar (hydrophilic) residue such as between arginine and lysine, between glutamine and asparagine, and between threonine and serine. Additionally, the substitution of a basic residue such as lysine, arginine or histidine for another or the substitution of an acidic residue such as aspartic acid or glutamic acid for another is also contemplated. Exemplary conservative amino acids substitution groups are: valine-leucine-isoleucine, phenylalanine-tyrosine, lysine-arginine, alanine-valine, and asparagine-glutamine.

The term biologically active peptide or protein analog further includes modified forms of a native peptide or protein incorporating stereoisomers (e.g., D-amino acids) of the twenty conventional amino acids, or unnatural amino acids such as α,α-disubstituted amino acids, N-alkyl amino acids, lactic acid. These and other unconventional amino acids may also be substituted or inserted within native peptides and proteins useful within the invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, ω-N-methylarginine, and other similar amino acids and imino acids (e.g., 4-hydroxyproline). In addition, biologically active peptide or protein analogs include single or multiple substitutions, deletions and/or additions of carbohydrate, lipid and/or proteinaceous moieties that occur naturally or artificially as structural components of the subject peptide or protein, or are bound to or otherwise associated with the peptide or protein.

In one aspect, peptides (including polypeptides) useful within the invention are modified to produce peptide mimetics by replacement of one or more naturally occurring side chains of the 20 genetically encoded amino acids (or D amino acids) with other side chains, for instance with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered heterocyclics. For example, proline analogs can be made in which the ring size of the proline residue is changed from 5 members to 4, 6, or 7 members. Cyclic groups can be saturated or unsaturated, and if unsaturated, can be aromatic or non-aromatic. Heterocyclic groups can contain one or more nitrogen, oxygen, and/or sulphur heteroatoms. Examples of such groups include the furazanyl, furyl, imidazolidinyl, imidazolyl, imidazolinyl, isothiazolyl, isoxazolyl, morpholinyl (e.g., morpholino), oxazolyl, piperazinyl (e.g., 1-piperazinyl), piperidyl (e.g., 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g., 1-pyrrolidinyl), pyrrolinyl, pyrrolyl, thiadiazolyl, thiazolyl, thienyl, thiomorpholinyl (e.g., thiomorpholino), and triazolyl. These heterocyclic groups can be substituted or unsubstituted. Where a group is substituted, the substituent can be alkyl, alkoxy, halogen, oxygen, or substituted or unsubstituted phenyl.

Peptides and proteins, as well as peptide and protein analogs and mimetics, can also be covalently bound to one or more of a variety of nonproteinaceous polymers, e.g., polyethylene glycol, polypropylene glycol, or polyoxyalkenes, in the manner set forth in U.S. Pat. No. 4,640,835; U.S. Pat. No. 4,496,689; U.S. Pat. No. 4,301,144; U.S. Pat. No. 4,670,417; U.S. Pat. No. 4,791,192; or U.S. Pat. No. 4,179,337.

Other peptide and protein analogs and mimetics within the invention include glycosylation variants, and covalent or aggregate conjugates with other chemical moieties. Covalent derivatives can be prepared by linkage of functionalities to groups which are found in amino acid side chains or at the N- or C-termini, by means which are well known in the art. These derivatives can include, without limitation, aliphatic esters or amides of the carboxyl terminus, or of residues containing carboxyl side chains, O-acyl derivatives of hydroxyl group-containing residues, and N-acyl derivatives of the amino terminal amino acid or amino-group containing residues, e.g., lysine or arginine. Acyl groups are selected from the group of alkyl-moieties including C3 to C18 normal alkyl, thereby forming alkanoyl aroyl species. Covalent attachment to carrier proteins, e.g., immunogenic moieties may also be employed.

In addition to these modifications, glycosylation alterations of biologically active peptides and proteins can be made, e.g., by modifying the glycosylation patterns of a peptide during its synthesis and processing, or in further processing steps. Particularly preferred means for accomplishing this are by exposing the peptide to glycosylating enzymes derived from cells that normally provide such processing, e.g., mammalian glycosylation enzymes. Deglycosylation enzymes can also be successfully employed to yield useful modified peptides and proteins within the invention. Also embraced are versions of a native primary amino acid sequence which have other minor modifications, including phosphorylated amino acid residues, e.g., phosphotyrosine, phosphoserine, or phosphothreonine, or other moieties, including ribosyl groups or cross-linking reagents.

Peptidomimetics may also have amino acid residues that have been chemically modified by phosphorylation, sulfonation, biotinylation, or the addition or removal of other moieties, particularly those that have molecular shapes similar to phosphate groups.

One can cyclize active peptides for use within the invention, or incorporate a desamino or descarboxy residue at the termini of the peptide, so that there is no terminal amino or carboxyl group, to decrease susceptibility to proteases, or to restrict the conformation of the peptide. C-terminal functional groups among peptide analogs and mimetics of the present invention include amide, amide lower alkyl, amide di(lower alkyl), lower alkoxy, hydroxy, and carboxy, and the lower ester derivatives thereof, and the pharmaceutically acceptable salts thereof.

A variety of additives, diluents, bases and delivery vehicles are provided within the invention that effectively control water content to enhance protein stability. These reagents and carrier materials effective as anti-aggregation agents in this sense include, for example, polymers of various functionalities, such as polyethylene glycol, dextran, diethylaminoethyl dextran, and carboxymethyl cellulose, which significantly increase the stability and reduce the solid-phase aggregation of peptides and proteins admixed therewith or linked thereto. In some instances, the activity or physical stability of proteins can also be enhanced by various additives to aqueous solutions of the peptide or protein drugs. For example, additives, such as polyols (including sugars), amino acids, proteins such as collagen and gelatin, and various salts may be used.

Certain additives, in particular sugars and other polyols, also impart significant physical stability to dry, e.g., lyophilized proteins. These additives can also be used within the invention to protect the proteins against aggregation not only during lyophilization but also during storage in the dry state. For example sucrose and Ficoll 70 (a polymer with sucrose units) exhibit significant protection against peptide or protein aggregation during solid-phase incubation under various conditions. These additives may also enhance the stability of solid proteins embedded within polymer matrices.

Yet additional additives, for example sucrose, stabilize proteins against solid-state aggregation in humid atmospheres at elevated temperatures, as may occur in certain sustained-release formulations of the invention. Proteins such as gelatin and collagen also serve as stabilizing or bulking agents to reduce denaturation and aggregation of unstable proteins in this context. These additives can be incorporated into polymeric melt processes and compositions within the invention. For example, polypeptide microparticles can be prepared by simply lyophilizing or spray drying a solution containing various stabilizing additives described above. Sustained release of unaggregated peptides and proteins can thereby be obtained over an extended period of time.

Various additional preparative components and methods, as well as specific formulation additives, are provided herein which yield formulations for mucosal delivery of aggregation-prone peptides and proteins, wherein the peptide or protein is stabilized in a substantially pure, unaggregated form. A range of components and additives are contemplated for use within these methods and formulations. Exemplary of these anti-aggregation agents are linked dimers of cyclodextrins (CDs), which selectively bind hydrophobic side chains of polypeptides. These CD dimers have been found to bind to hydrophobic patches of proteins in a manner that significantly inhibits aggregation. This inhibition is selective with respect to both the CD dimer and the protein involved. Such selective inhibition of protein aggregation provides additional advantages within the intranasal delivery methods and compositions of the invention. Additional agents for use in this context include CD trimers and tetramers with varying geometries controlled by the linkers that specifically block aggregation of peptides and proteins (Breslow, et al., J. Am. Chem. Soc. 118:11678-11681, 1996; Breslow, et al., PNAS USA 94:11156-11158, 1997).

Charge Modifying and pH Control Agents and Methods

To improve the transport characteristics of biologically active agents (e.g., macromolecular drugs, peptides or proteins) for enhanced delivery across hydrophobic mucosal membrane barriers, the invention also provides techniques and reagents for charge modification of selected biologically active agents or delivery-enhancing agents described herein. In this regard, the relative permeabilities of macromolecules is generally be related to their partition coefficients. The degree of ionization of molecules, which is dependent on the pKₐ of the molecule and the pH at the mucosal membrane surface, also affects permeability of the molecules. Permeation and partitioning of biologically active agents and permeabilizing agents for mucosal delivery may be facilitated by charge alteration or charge spreading of the active agent or permeabilizing agent, which is achieved, for example, by alteration of charged functional groups, by modifying the pH of the delivery vehicle or solution in which the active agent is delivered, or by coordinate administration of a charge- or pH-altering reagent with the active agent.

Preservatives

Preservative such as chlorobutanol, methyl paraben, propyl paraben, sodium benzoate (0.5%), phenol, cresol, p-chloro-m-cresol, phenylethyl alcohol, benzyl alcohol, phenylmercuric acetate, phenylmercuric borate, phenylmercuric nitrate, thimerosal, sorbic acid, benzethonium chloride or benzylkonium chloride can be added to the formulations of the invention to inhibit microbial growth.

pH and Buffering Systems

The pH is generally regulated using a buffer such as a system comprised of citric acid and a citrate salt(s), such as sodium citrate. Additional suitable buffer systems include acetic acid and an acetate salt system, succinic acid and a succinate salt system, malic acid and a malic salt system, and gluconic acid and a gluconate salt system. Alternatively, buffer systems comprised of mixed acid/salt systems can be employed, such as an acetic acid and sodium citrate system, a citrate acid, sodium acetate system, and a citric acid, sodium citrate, sodium benzoate system. For any buffer system, additional acids, such as hydrochloric acid, and additional bases, such as sodium hydroxide, may be added for final pH adjustment.

Additional Agents for Modulating Epithelial Junction Structure and/or Physiology

Epithelial tight junctions are generally impermeable to molecules with radii of approximately 15 angstroms, unless treated with junctional physiological control agents that stimulate substantial junctional opening as provided within the instant invention. Among the “secondary” tight junctional regulatory components that will serve as useful targets for secondary physiological modulation within the methods and compositions of the invention, the ZO1-ZO2 heterodimeric complex has shown itself amenable to physiological regulation by exogenous agents that can readily and effectively alter paracellular permeability in mucosal epithelia. On such agent that has been extensively studied is the bacterial toxin from Vibrio cholerae known as the “zonula occludens toxin” (ZOT). See also, WO 96/37196; U.S. Pat. Nos. 5,945,510; 5,948,629; 5,912,323; 5,864,014; 5,827,534; 5,665,389; and 5,908,825. Thus, ZOT and other agents that modulate the ZO1-ZO2 complex will be combinatorially formulated or coordinately administered with one or more biologically active agents.

Formulation and Administration

Mucosal delivery formulations of the present invention comprise the biologically active agent to be administered typically combined together with one or more pharmaceutically acceptable carriers and, optionally, other therapeutic ingredients. The carrier(s) must be “pharmaceutically acceptable” in the sense of being compatible with the other ingredients of the formulation and not eliciting an unacceptable deleterious effect in the subject. Such carriers are described herein above or are otherwise well known to those skilled in the art of pharmacology. Desirably, the formulation should not include substances such as enzymes or oxidizing agents with which the biologically active agent to be administered is known to be incompatible. The formulations may be prepared by any of the methods well known in the art of pharmacy.

The compositions and methods of the invention may be administered to subjects by a variety of mucosal administration modes, including by oral, rectal, vaginal, intranasal, intrapulmonary, or transdermal delivery, or by topical delivery to the eyes, ears, skin or other mucosal surfaces. Compositions according to the present invention are often administered in an aqueous solution as a nasal or pulmonary spray and may be dispensed in spray form by a variety of methods known to those skilled in the art. Preferred systems for dispensing liquids as a nasal spray are disclosed in U.S. Pat. No. 4,511,069. Such formulations may be conveniently prepared by dissolving compositions according to the present invention in water to produce an aqueous solution, and rendering said solution sterile. The formulations may be presented in multi-dose containers, for example in the sealed dispensing system disclosed in U.S. Pat. No. 4,511,069. Other suitable nasal spray delivery systems have been described in Transdermal Systemic Medication, Y. W. Chien ed., Elsevier Publishers, New York, 1985; and in U.S. Pat. No. 4,778,810. Additional aerosol delivery forms may include, e.g., compressed air-, jet-, ultrasonic-, and piezoelectric nebulizers, which deliver the biologically active agent dissolved or suspended in a pharmaceutical solvent, e.g., water, ethanol, or a mixture thereof.

To formulate compositions for mucosal delivery within the present invention, the biologically active agent can be combined with various pharmaceutically acceptable additives, as well as a base or carrier for dispersion of the active agent(s). Desired additives include, but are not limited to, pH control agents, such as arginine, sodium hydroxide, glycine, hydrochloric acid, citric acid, etc. In addition, local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione) can be included. When the composition for mucosal delivery is a liquid, the tonicity of the formulation, as measured with reference to the tonicity of 0.9% (w/v) physiological saline solution taken as unity, is typically adjusted to a value at which no substantial, irreversible tissue damage will be induced in the nasal mucosa at the site of administration. Generally, the tonicity of the solution is adjusted to a value of about ⅓ to 3, more typically ½ to 2, and most often ¾ to 1.7.

The biologically active agent may be dispersed in a base or vehicle, which may comprise a hydrophilic compound having a capacity to disperse the active agent and any desired additives. The base may be selected from a wide range of suitable carriers, including but not limited to, copolymers of polycarboxylic acids or salts thereof, carboxylic anhydrides (e.g., maleic anhydride) with other monomers (e.g., methyl (meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters, etc., can be employed as carriers. Hydrophilic polymers and other carriers can be used alone or in combination, and enhanced structural integrity can be imparted to the carrier by partial crystallization, ionic bonding, crosslinking and the like. The carrier can be provided in a variety of forms, including, fluid or viscous solutions, gels, pastes, powders, microspheres and films for direct application to the nasal mucosa. The use of a selected carrier in this context may result in promotion of absorption of the biologically active agent.

The biologically active agent can be combined with the base or carrier according to a variety of methods, and release of the active agent may be by diffusion, disintegration of the carrier, or associated formulation of water channels. In some circumstances, the active agent is dispersed in microcapsules (microspheres) or nanocapsules (nanospheres) prepared from a suitable polymer, e.g., isobutyl 2-cyanoacrylate (see, e.g., Michael, et al., J. Pharmacy Pharmacol. 43:1-5, 1991), and dispersed in a biocompatible dispersing medium applied to the nasal mucosa, which yields sustained delivery and biological activity over a protracted time.

To further enhance mucosal delivery of pharmaceutical agents within the invention, formulations comprising the active agent may also contain a hydrophilic low molecular weight compound as a base or excipient. Such hydrophilic low molecular weight compounds provide a passage medium through which a water-soluble active agent, such as a physiologically active peptide or protein, may diffuse through the base to the body surface where the active agent is absorbed. The hydrophilic low molecular weight compound optionally absorbs moisture from the mucosa or the administration atmosphere and dissolves the water-soluble active peptide. The molecular weight of the hydrophilic low molecular weight compound is generally not more than 10,000 and preferably not more than 3,000. Exemplary hydrophilic low molecular weight compound include polyol compounds, such as oligo-, di- and monosaccarides such as sucrose, mannitol, lactose, L-arabinose, D-erythrose, D-ribose, D-xylose, D-mannose, D-galactose, lactulose, cellobiose, gentibiose, glycerin and polyethylene glycol. Other examples of hydrophilic low molecular weight compounds useful as carriers within the invention include N-methylpyrrolidone, and alcohols (e.g., oligovinyl alcohol, ethanol, ethylene glycol, propylene glycol, etc.). These hydrophilic low molecular weight compounds can be used alone or in combination with one another or with other active or inactive components of the intranasal formulation.

The compositions of the invention may alternatively contain as pharmaceutically acceptable carriers substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

In certain embodiments of the invention, the biologically active agent is administered in a time release formulation, for example in a composition which includes a slow release polymer. The active agent can be prepared with carriers that will protect against rapid release, for example a controlled release vehicle such as a polymer, microencapsulated delivery system or bioadhesive gel. Prolonged delivery of the active agent, in various compositions of the invention can be brought about by including in the composition agents that delay absorption, for example, aluminum monosterate hydrogels and gelatin.

The term “subject” as used herein means any mammalian patient to which the compositions of the invention may be administered.

Kits

The instant invention also includes kits, packages and multicontainer units containing the above described pharmaceutical compositions, active ingredients, and/or means for administering the same for use in the prevention and treatment of diseases and other conditions in mammalian subjects. Briefly, these kits include a container or formulation that contains one or more biologically active agent formulated in a pharmaceutical preparation for mucosal delivery. The biologically active agent(s) is/are optionally contained in a bulk dispensing container or unit or multi-unit dosage form. Optional dispensing means may be provided, for example a pulmonary or intranasal spray applicator. Packaging materials optionally include a label or instruction indicating that the pharmaceutical agent packaged therewith can be used mucosally, e.g., intranasally, for treating or preventing a specific disease or condition.

The above disclosure generally describes the present invention, which is further exemplified by the following examples. These examples are described solely for purposes of illustration, and are not intended to limit the scope of the invention. Although specific terms and values have been employed herein, such terms and values will likewise be understood as exemplary and non-limiting to the scope of the invention.

Polynucleotide Delivery Enhancing Polypeptides

Within additional embodiments of the invention, the polynucleotide delivery-enhancing polypeptide is selected or rationally designed to comprise an amphipathic amino acid sequence. For example, useful polynucleotide delivery-enhancing polypeptides may be selected which comprise a plurality of non-polar or hydrophobic amino acid residues that form a hydrophobic sequence domain or motif, linked to a plurality of charged amino acid residues that form a charged sequence domain or motif, yielding an amphipathic peptide.

In other embodiments, the polynucleotide delivery-enhancing polypeptide is selected to comprise a protein transduction domain or motif, and a fusogenic peptide domain or motif. A protein transduction domain is a peptide sequence that is able to insert into and preferably transit through the membrane of cells. A fusogenic peptide is a peptide that is able destabilize a lipid membrane, for example a plasma membrane or membrane surrounding an endosome, which may be enhanced at low pH. Exemplary fusogenic domains or motifs are found in a broad diversity of viral fusion proteins and in other proteins, for example fibroblast growth factor 4 (FGF4).

To rationally design polynucleotide delivery-enhancing polypeptides of the invention, a protein transduction domain is employed as a motif that will facilitate entry of the nucleic acid into a cell through the plasma membrane. In certain embodiments, the transported nucleic acid will be encapsulated in an endosome. The interior of endosomes has a low pH resulting in the fusogenic peptide motif destabilizing the membrane of the endosome. The destabilization and breakdown of the endosome membrane allows for the release of the siNA into the cytoplasm where the siNA can associate with a RISC complex and be directed to its target mRNA.

Examples of protein transduction domains for optional incorporation into polynucleotide delivery-enhancing polypeptides of the invention include:

TAT protein transduction domain (PTD)
(SEQ ID NO: 1) KRRQRRR;
Penetratin PTD (SEQ ID NO: 2) RQIKIWFQNRRMKWKK;
VP22 PTD (SEQ ID NO: 3)
DAATATRGRSAASRPTERPRAPARSASRPRRPVD;
Kaposi FGF signal sequences (SEQ ID NO: 4)
AAVALLPAVLLALLAP, and SEQ ID NO: 5)
AAVLLPVLLPVLLAAP;
Human β3 integrin signal sequence (SEQ ID NO: 6)
VTVLALGALAGVGVG;
gp41 fusion sequence (SEQ ID NO: 7)
GALFLGWLGAAGSTMGA;
Caiman crocodylus Ig(v) light chain (SEQ ID NO: 8)
MGLGLHLLVLAAALQGA;
hCT-derived peptide (SEQ ID NO: 9)
LGTYTQDFNKFHTFPQTAIGVGAP;
Transportan (SEQ ID NO: 10)
GWTLNSAGYLLKINLKALAALAKKIL;
Loligomer (SEQ ID NO: 11) TPPKKKRKVEDPKKKK;
Arginine peptide (SEQ ID NO: 12) RRRRRRR;
and
Amphiphilic model peptide (SEQ ID NO: 13)
KLALKLALKALKAALKLA.

Examples of viral fusion peptides fusogenic domains for optional incorporation into polynucleotide delivery-enhancing polypeptides of the invention include:

Influenza HA2 (SEQ ID NO: 14) GLFGAIAGFIENGWEG;
Sendai F1 (SEQ ID NO: 15) FFGAVIGTIALGVATA;
Respiratory Syncytial virus F1 (SEQ ID NO: 16)
FLGFLLGVGSAIASGV;
HIV gp41 (SEQ ID NO: 17) GVFVLGFLGFLATAGS;
and
Ebola GP2 (SEQ ID NO: 18) GAAIGLAWIPYFGPAA.

Within yet additional embodiments of the invention, polynucleotide delivery-enhancing polypeptides are provided that incorporate a DNA-binding domain or motif which facilitates polypeptide-siNA complex formation and/or enhances delivery of siNAs within the methods and compositions of the invention. Exemplary DNA binding domains in this context include various “zinc finger” domains as described for DNA-binding regulatory proteins and other proteins identified below (see, e.g., Simpson, et al., J. Biol. Chem. 278:28011-28018, 2003).

TABLE 1
Exemplary Zinv Finger Motifs of Different DNA-Binding Proteins

C₂H₂ Zinc finger motif

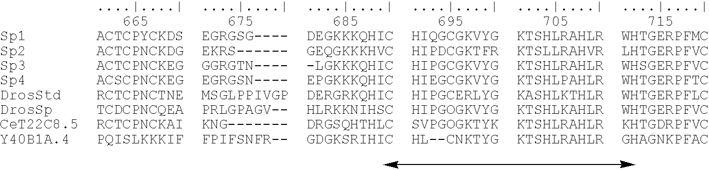

Prosite pattern
C-x(2,4)-C-x(12)-H-x(3)-H

Table 1 demonstrates a conservative zinc fingerer motif for double strand DNA binding which is characterized by the C-x(2,4)-C-x(12)-H-x(3)-H motif pattern, which itself can be used to select and design additional polynucleotide delivery-enhancing polypeptides according to the invention.

The sequences shown in Table 1, for Sp1, Sp2, Sp3, Sp4, DrosBtd, DrosSp, CeT22C8.5, and Y4pB1A.4, are herein assigned SEQ ID NOs: 19, 20, 21, 22, 23, 24, 25, and 26, respectively.

Alternative DNA binding domains useful for constructing polynucleotide delivery-enhancing polypeptides of the invention include, for example, portions of the HIV Tat protein sequence (see Examples below).

Within exemplary embodiments of the invention described herein below, polynucleotide delivery-enhancing polypeptides may be rationally designed and constructed by combining any of the foregoing structural elements, domains or motifs into a single polypeptide effective to mediate enhanced delivery of siNAs into target cells. For example, a protein transduction domain of the TAT polypeptide was fused to the N-terminal 20 amino acids of the influenza virus hemagglutinin protein, termed HA2, to yield one exemplary polynucleotide delivery-enhancing polypeptide herein. Various other polynucleotide delivery-enhancing polypeptide constructs are provided in the instant disclosure, evincing that the concepts of the invention are broadly applicable to create and use a diverse assemblage of effective polynucleotide delivery-enhancing polypeptides for enhancing siNA delivery.

Yet additional exemplary polynucleotide delivery-enhancing polypeptides within the invention may be selected from the following peptides:

| | |
|---|---|
| WWETWKPFQCRICMRNFSTRQARRNHRRRHR; | (SEQ ID NO: 27) |
| GKINLKALAALAKKIL, | (SEQ ID NO: 28) |
| RVIRVWFQNKRCKDKK, | (SEQ ID NO: 29) |
| GRKKRRQRRRPPQGRKKRRQRRRPPQGRKKRRQR | (SEQ ID NO: 30) |
| RRPPQ, | |
| GEQIAQLIAGYIDIILKKKKSK, | (SEQ ID NO: 31) |

Poly Lys-Trp, 4:1, MW 20,000-50,000; and

Poly Orn-Trp, 4:1, MW 20,000-50,000.

Additional polynucleotide delivery-enhancing polypeptides that are useful within the compositions and methods herein comprise all or part of the mellitin protein sequence.

All publications, references, patents, patent publications and patent applications cited herein are each hereby specifically incorporated by reference in their entirety.

While this invention has been described in relation to certain embodiments, and many details have been set forth for purposes of illustration, it will be apparent to those skilled in the art that this invention includes additional embodiments, and that some of the details described herein may be varied considerably without departing from this invention. This invention includes such additional embodiments, modifications and equivalents. In particular, this invention includes any combination of the features, terms, or elements of the various illustrative components and examples.

The use herein of the terms “a,” “an,” “the,” and similar terms in describing the invention, and in the claims, are to be construed to include both the singular and the plural. The terms “comprising,” “having,” “including,” and “containing” are to be construed as open-ended terms which mean, for example, “including, but not limited to.” Recitation of a range of values herein refers individually to each separate value falling within the range as if it were individually recited herein, whether or not some of the values within the range are expressly recited. Specific values employed herein will be understood as exemplary and not to limit the scope of the invention.

Definitions of technical terms provided herein should be construed to include without recitation those meanings associated with these terms known to those skilled in the art, and are not intended to limit the scope of the invention.

The examples given herein, and the exemplary language used herein are solely for the purpose of illustration, and are not intended to limit the scope of the invention.

When a list of examples is given, such as a list of compounds or molecules suitable for this invention, it will be apparent to those skilled in the art that mixtures of the listed compounds or molecules are also suitable.

### EXAMPLES

The invention will be further described in the following examples, which do not limit the scope of the invention described in the claims. The following examples describe tight junction modulating peptides (TJMPs) that enhance the permeation of therapeutics agents across an epithelial cell layer.

### Example 1

### In Vitro Methods and Protocols

Tight junction modulating peptides or TJMPs are peptides capable of compromising the integrity of tight junctions with the effect of creating openings between epithelial cells and thus reducing the barrier function of an epithelia. The state of tight junction integrity can be assayed in vitro by measuring the level of electrical resistance and degree sample permeation across a human nasal epithelial tissue model system. A reduction in electrical resistance and enhanced permeation suggests that the tight junctions have been compromised and openings have been created between the epithelial cells. In effect, peptides that induce a measured reduction in electrical resistance across a tissue membrane, referred to as (TER) reduction, and promote enhanced permeation of a small molecule through a tissue membrane are classified as TJMPs. In addition, the level of cell toxicity for TJMPs is also assessed to determine whether these peptides could function as tight junction modulating peptides in drug delivery across a mucosal surface, for example intranasal (IN) drug delivery.

The assays used to screen the exemplary peptides of the present invention (refer to Table 3 of Example 2) are described in the present example. These assays include transepithelial electrical resistance (TER), cytotoxicity (LDH), and sample permeation. Also described are the reagents used and the cell culture conditions.

Table 2 illustrates the sample reagents used in the subsequent Examples.

TABLE 2
Sample Reagents

| Reagent | Manufacturer |
|---|---|
| 1× DPBS++ | Gibco/Invitrogen ™ |
| Sterile, Nulcease-Free Water | Ambion ™ |
| Fluorescent Dextran | Molecular Probes/Invitrogen ™ |
| Air-100 Medium ™ | MatTek ™ |
| Air-196 inserts ™ | MatTek ™ |
| CytoTox 96 Assay ™ | Promega ™ |

Cell Culture

The EpiAirway™ system was developed by MatTek Corp. (Ashland, Mass.) as a model of the pseudostratified epithelium lining the respiratory tract. The epithelial cells are grown on porous membrane-bottomed cell culture inserts at an air-liquid interface, which results in differentiation of the cells to a highly polarized morphology. The apical surface is ciliated with a microvillous ultrastructure and the epithelium produces mucus (the presence of mucin has been confirmed by immunoblotting). The cells are plated onto the inserts at the factory approximately three weeks before shipping.

EpiAirway™ culture membranes were received the day before the experiments started. They are shipped in phenol red-free and hydrocortisone-free Dulbecco's Modified Eagle's Medium (DMEM). The cells are ciliated and psudostratefied, grown to confluency on Millipore Multiscreen Caco-2 96-well assay system comprised of a polycarbonate filter system. Upon receipt, the insert system will be stored unopened at 4° C. and/or cultured in 250 μl basal media per well (phenol red-free and hydrocortisone-free Dulbecco's Modified Eagle's Medium (DMEM)) at 37° C./5% CO₂ for 24 hours before use.

This model system was used to evaluate the efficacy of TJMPs to modulate TEER, effect cytotoxicity and enhance permeation of an epithelial cell monolayer.

Peptide Synthesis

Peptide syntheses were performed on a Rainin Symphony synthesizer on a 50 umol scale using NovaBiochem TGR resin. Deprotections were performed by two treatments of 20% piperidine in DMF for 10 minutes. After deprotection the resin was washed once with 10 mL DMF containing 5% HOBt (30 s) and 4 times with 10 mL DMF (30 s). Couplings were performed by delivering 5-fold excess Fmoc amino acid in DMF to the reaction vessel followed by delivery of an equal volume of activator solution containing 6.25-fold excess N-methylmorpholine and 5-fold excess of HCTU. A coupling time of 40 minutes was used throughout the synthesis. After the first coupling reaction the resin was washed twice with 10 mL of DMF (30 s) prior to initiating the second coupling step. For pegylated peptides, upon completion of the peptide synthesis the N-terminal Fmoc group was removed and 2 equivalents of O-(N-Fmoc-2-aminoethyl)-O′-(2-carboxyethyl)-undecaethyleneglycol in DMF were added manually to the reaction vessels. While in manual mode, 2 equivalents of activator solution were delivered to the reaction vessel and the coupling was allowed to proceed overnight. Generally, coupling efficiencies of greater than 97% was achieved and any unreacted peptide was capped by acetic anhydride.

Cleavage was performed on the individual reaction vessels by delivery of 10 mL of TFA containing 2.5% TIS, 2.5% water followed by gentle nitrogen agitation for 3 hour. The cleavage solution was collected automatically into conical tubes, pooled and the volume was reduced by evaporation under reduced pressure. The resulting solution was triturated with an excess of cold ether, filtered and washed extensively with cold ether. After drying, the crude peptide was taken up in Millipore water and lyophilized to dryness.

FITC (fluorescein-5-isothiocyanate)-Dextran Permeation Assay

A FITC labeled dextran with a molecular weight 3000 (FD3) was used to assess the efficacy of individual TJMP on epithelial cell monolayer permeation. The tissue insert plates were transferred to a 96-well receiver plate containing 200 μl of DPBS++ as basal media. The apical surface of each tissue culture insert was incubated with a 20 μl sample of a single test formulation (refer to Table 4 in Example 2 for details of test formulations) for one hour at 37° C. in the dark on a shaker (˜100 rpm). Following the 1 hour incubation period, underlying basal media samples were taken from each tissue culture insert and temporarily stored in the dark at room temperature until FD3 levels were quantified by fluorescence spectroscopy. For FD3 measurements, a 150 μl of basal media sample was transferred to a black, clear bottom 96-well plate. Fluorescence emission at 528/20 following excitation at 485/20 were measured using a FLx800 fluorescence plate reader from Biotek Instruments.

Permeation was calculated as:

$$\% Permeation = \frac{Cb \times Vb}{Ca \times Va} \times 100$$

$$Apparent Permeability , cm \text{/} sec ( {P}_{app} ) = \frac{Vb}{SA \times Ca} \frac{Cb}{dt}$$

Formula terms for permeation defined:

Cb: Basolateral concentration

Ca: Apical Concentration

Vb: Basolateral Volume

Va: Apical Volume

SA: Filter Surface Area

dt: Elapsed Time

Transepithelial Electrical Resistance (TER) and TER Recovery

TER measurements were taken using the REMS Autosampler (World Precision Instruments, Sarasota, Fla.) with the electrode leads. The electrodes and a tissue culture blank insert will be equilibrated for at least 20 minutes in MatTek Air-100™ medium with the power off prior to checking calibration. The background resistance of the insert system has been established by multiple measurements of a blank insert plate and the same value was used for each test on the platform. Time zero TER (TER0) was measured before incubation of the inserts with the test formulation. The top electrode will be as adjusted so that it is close to, but not making contact with, the top surface of the insert membrane. Background resistance of the blank insert should be about 5-20 ohms. For each TER determination, 100 μl of MatTek Air-100™ medium was added to the insert and 250 μl in the basal well followed by placement in the Endohm chamber. All TER values are reported as a function of the surface area of the tissue. Resistance was expressed as both Ohms*cm² and percent original TER value.

TER values were calculated as:

$$Nominal Resistance , Ohm * {cm}^{2} = ( TERt - blank ) * 0.12$$

$$Relative TER , \% = \frac{TERt - blank}{TER 0 - blank} \times 100$$

Formula terms for TER calculation defined:

TER0: TER measurement at time zero.

TERt: TER measurement taken at time t after test formulation incubation

blank: Background resistance measurement

A decrease in TER value relative to the control value indicates a decrease in cell membrane resistance and an increase in mucosal epithelial cell permeability.

Cytotoxicity (LDH Assay)

The amount of cell death was assayed by measuring the release of lactate dehydrogenase (LDH) from the cells into the apical medium using a CytoTox 96 Cytotoxicity Assay Kit (Promega Corp., Madison, Wis.). One percent Octylphenolpoly (ethyleneglycolether)×(Triton X-100™) diluted in phosphate buffered saline (PBS) causes 100% lysis in cultured cells and served herein as a positive control for the LDH assay. Following the one hour incubation period with a test formulation (refer to Table 4 in Example 2 for details of test formulations), the total liquid volume of each insert was brought to a final volume of 200 μl with culture medium. The apical medium was then mixed by pipetting four times with a multichannel pipette set to a 100 μl volume. After mixing, a 100 μl sample from the apical side of each insert was transferred to a new 96-well plate. The apical media samples were sealed with a plate sealer and stored at room temperature for same day analysis or stored overnight at 4° C. for analysis the next day. To measure LDH levels, 5 μl of the 100 μl apical media sample was diluted in 45 μl DPBS in a new 96-well plate. Fresh, cell-free culture medium will be used as a blank. Fifty microliters of substrate solution was added to each well and incubated for 30 minutes at room temperature away from direct light. Following the 30 minute incubation, 50 μl of stop solution was added to each well. Optical density (OD) was measured at 490 nm with a uQuant absorbance plate reader from Biotek Instruments. The measurement of LDH release into the apical media indicates relative cytotoxicity of the samples. Percent cytotoxicity for each test formulation was calculated by subtracting the measured absorbance of the PBS control (basal level of LDH release) from the measured absorbance of the individual test formulation and then dividing that value by the measured absorbance for the 1% Triton X-100™ positive control, multiplied by 100.

The formula used to calculate percent cytotoxicity is as follows:

$$Relative Cytotoxicity , \% = \frac{ODx - ODpbs}{ODtriton} \times 100$$

Osmolality

Samples were measured by Model 20200 from Advanced Instruments Inc. (Norwood, Mass.).

### Example 2

### Tight Junction Modulating Peptides that Enhance Epithelial Cell Layer Permeation In Vitro

The present example describes the exemplary peptides PN679 and PN745 of the present invention (shown in Table 3) and the test formulation for each peptide (shown in Table 4) screened to determine each peptide's effective concentration range for epithelial cell monolayer permeation enhancement.

TABLE 3

Tight Junction Modulating Peptides

| | |
|---|---|
| Peptide | Molecular |

| | | |
|---|---|---|
| # | Amino Acid Sequence | Weight |

| | | |
|---|---|---|
| PN679 | CNGRCGGKKKLKLLLKLL | 1984.78 |
| | (SEQ ID NO: 32) | |
| PN745 | LRKLRKRLLRLRKLRKRLLR-amide | 2684.53 |
| | (SEQ ID NO: 33) | |

Table 4 below describes the individual test formulations containing an exemplary peptide (“Active Agent” column in Table 4) of the present invention and the test formulations that served as either a positive and negative test formulation controls that were examined by TER, LDH (cytotoxicity) and sample permeation enhancement assays. Each peptide was tested at a 25 μM, 100 μM, 250 μM, 500 μM and 1000 μM concentration. PN159 (test formulation #11) herein served as a TJMP positive control and has previously demonstrated the ability to effectively reduce TER and enhance sample permeation at 25 μM. One percent Triton X-100™ (test formulation #14) functioned as a positive control for both the cytotoxicity (LDH) assay and TER reduction assay. Peptide Delivery Formulation (“PDF”) served herein as a small molecule permeation enhancer. The DPBS++ served as a negative control. Each test formulation had a final volume of 300 μl and a target pH of 7 except test formulation #12, which had a target pH of 5. One percent Triton X-100™ (test formulation #14) functioned as a positive (i.e., 100% release) control for the cytotoxicity (LDH) assay.

Of the total 300 μl volume for each test formulation, only a 20 μl sample was applied to the human-derived tracheal/bronchial epithelial cells (EpiAirway™ Tissue model system) in order to assess the effect each test formulation had on TER, LDH and sample permeation.

TABLE 4
Test Formulations

| Test | | | | | | | | | | | |
|---|---|---|---|---|---|---|---|---|---|---|---|
| Formulation | Active | Treatment | | | | DPBS++ | | Active | | | |
| # | Agent | Concentration | | Water | | (pH 7.5) | | Agent | | 10× FD3 | |
| 1 | PN679 | 1000 | μM | 15 | μl | 225 | μl | 30 | μl | 30 | μl |
| 2 | | 500 | μM | 30 | μl | 225 | μl | 15 | μl | 30 | μl |
| 3 | | 250 | μM | 37.5 | μl | 225 | μl | 7.5 | μl | 30 | μl |
| 4 | | 100 | μM | 42 | μl | 225 | μl | 3 | μl | 30 | μl |
| 5 | | 25 | μM | 44.3 | μl | 225 | μl | 0.75 | μl | 30 | μl |
| 6 | PN745 | 1000 | μM | 15 | μl | 225 | μl | 30 | μl | 30 | μl |
| 7 | | 500 | μM | 30 | μl | 225 | μl | 15 | μl | 30 | μl |
| 8 | | 250 | μM | 44.9 | μl | 225 | μl | 0.075 | μl | 30 | μl |
| 9 | | 100 | μM | 44.97 | μl | 225 | μl | 0.03 | μl | 30 | μl |
| 10 | | 25 | μM | 44.3 | μl | 225 | μl | 0.75 | μl | 30 | μl |
| 11 | PN159 | 25 | μM | 43.9 | μl | 225 | μl | 1.1 | μl | 30 | μl |

| | | | | | | | | | | |
|---|---|---|---|---|---|---|---|---|---|---|
| 12 | PDF | 1× | 120 | μl | 0 | μl | 150 | μl | 30 | μl |
| 13 | | 0.75× DPBS++ | 45 | μl | 225 | μl | 0 | μl | 30 | μl |
| 14 | | 1% Triton | 41.7 | μl | 225 | μl | 33.33 | μl | 0 | μl |
| | | X-100 ™ | | | | | | | | |

### Example 3

### PN679 and PN745 Modulate Tight Junction Proteins In Vitro

The present example demonstrates that the exemplary peptides PN679 and PN745 effectively reduced TER and significantly enhanced sample permeation in a dose-dependent manner without causing significant cell toxicity indicating that these peptides are effective TJMPs. Table 5 summarizes the TER, LDH and sample permeation (FD3) data for the test formulations described in Table 4 of Example 2. Test formulation #1 for PN679 and test formulations #6 for PN745 were assayed twice.

TABLE 5
Summary of TER, LDH and Sample
Permeation Enhancement Data

| Test | | | | % FD3 |
|---|---|---|---|---|
| Formulation # | Active Agent | % T0 TER | LDH | Permeation |
| 1 | PN679 | −2% | 51% | 10% |
| 2 | | −2% | 50% | 10% |
| 3 | | 2% | 38% | 8% |
| 4 | | 7% | 23% | 7% |
| 5 | | 70% | 1% | 0% |
| 6 | PN745 | −3% | 45% | 7% |
| 7 | | 1% | 45% | 7% |
| 8 | | 1% | 45% | 8% |
| 9 | | 7% | 28% | 6% |
| 10 | | 24% | 11% | 2% |
| 11 | PN159 | 7% | 31% | 8% |
| | (Peptide Control) | | | |
| 12 | PDF | −2% | 27% | 18% |
| 13 | DPBS++ | 91% | 0% | 0% |
| 14 | Triton X-100 ™ | | 100% | |

The test formulations including 100 μM, 250 μM, 500 μM and 1000 μM of either of the exemplary peptides PN679 (test formulations #1, #2, #3 and #4) or PN745 (test formulations #6, #7, #8 and #9) of the present invention reduced TER to a degree equivalent to PDF and significantly below that of the established TJMP control PN159. As expected, the DPBS++ negative control did not reduce TER significantly. The ability of both these peptides to reduce TER correlated strongly with their ability to enhance permeation of the FD3 molecule. The 100 μM does for both PN679 (test formulation #4) and PN745 (test formulation #9) exhibited a percent permeation similar to the PN159 TJMP but with lower cytotoxicity (lower % LDH Release). Higher concentrations of either peptide resulted in increased levels of FD3 permeation above that of PN159, but also increased release of LDH levels indicating increased cytotoxicity. The DPBS++control did not induce a measurable LDH release. Based on the observed TER reduction, sample permeation and cytotoxicity (LDH release), a 100 μM concentration of either PN679 or PN745 appears optimal.

The foregoing data shows the unexpected discovery that the exemplary peptides PN679 and PN745 reduce TER and enhance small molecule permeation without significant toxicity of a human epithelial cell monolayer in vitro. These data indicate that these tight TMJPs are excellent candidates for use in drug delivery across a mucosal surface, for example intranasal (IN) drug delivery.

### Example 4

### Enhanced Permeation In Vitro by a Tight Junction Modulating Peptide Correlates Strongly with Enhanced Permeation Observed In Vivo

A linear regression analysis was performed to determine whether the TJMP permeation kinetics observed in the in vitro EpiAirway epithelial cell model system correlated with the in vivo pharmacokinetic data observed for that same TJMP. To determine if in vitro permeation data functions as a good indicator for success in vivo, the area under the curve-last value (AUC-last) derived from in vivo pharmacokinetic studies done with PYY and TJMPs was plotted against in vitro epithelial cell monolayer permeation studies done with PYY and TJMPs. In vitro permeation was expressed as a percentage and AUC-last as Min*pg/ml. In vitro and in vivo studies for 10 different TJMPs were graphed and a linear regression performed. An R² value of 0.82 (82% correlation) was derived indicating a strong correlation exist for AUC values derived in vivo and percent permeability observed in vitro. Surprisingly, when inter-assay variability is excluded, an R² value of 0.996 was derived indicating a direct correlation exist between in vitro permeability and in vivo success. Thus, in vitro permeation can be used to predict in vivo success.

### Example 5

### In Vivo Permeation Enhancement by a TJMP for a Peptide Hormone Therapeutic Agent Equals or Exceeds That of Small Molecule Permeation Enhancers

Twenty male New Zealand White rabbits age 3-6 months and weighing 2.1-3.0 kg were randomly assigned into one of 5 treatment groups with four animals per group. Test animals were dosed at 15 μl/kg and intranasally via pipette. Table 6 below indicates the composition of five different dose groups.

For dosing group 1 (see Table 6) a clinical formulation of PYY including small molecule permeation enhancers was used. The small molecule enhancers in these studies included methyl-β-cyclodextrin, phosphatidylcholine didecanoyl (DDPC), and/or EDTA. Dosing group 2 received PYY dissolved in phosphate buffered saline (PBS). For dosing groups 3-5, various concentrations of PN159 were added to dosing group 2, so that each of dosing groups 3 to 5 consisted of PYY, PN159, and PBS.

TABLE 6
Dosing Groups

| | Ani- | Permeation | Dose Conc | Dose Vol | PYY Dose |
|---|---|---|---|---|---|
| Group | mals | enhancers | (mg/ml) | (ml/kg) | (μg/kg) |
| 1 | 4M | Small molecule | 13.67 | 0.015 | 205 |
| | | permeation | | | |
| | | enhancers | | | |
| 2 | 4M | None | 13.67 | 0.015 | 205 |
| 3 | 4M | 25 μM PN159 | 13.67 | 0.015 | 205 |
| 4 | 4M | 50 μM PN159 | 13.67 | 0.015 | 205 |
| 5 | 4M | 100 μM PN159 | 13.67 | 0.015 | 205 |

Serial blood samples (about 2 ml each) were collected by direct venipuncture from a marginal ear vein into blood collection tubes containing EDTA as an anticoagulant. Blood samples were collected at 0, 2.5, 5, 10, 15, 30, 45, 60, and 120 minutes post-dosing. After collection of the blood, the tubes were gently rocked several times for anti-coagulation, and then 50 μl aprotinin solution was added. The blood was centrifuged at approximately 1,600×g for 15 minutes at approximately 4° C., and plasma samples were dispensed into duplicate aliquots and stored frozen at approximately −70° C.

Averaging all four animals in a treatment group, the following plasma concentrations of PYY were measured (Table 7):

TABLE 7
Summary of PYY Plasma Concentrations for Test Groups

| | Group 1 | Group 2 | | | |
|---|---|---|---|---|---|
| | Small molecule | No | Group 3 | Group 4 | Group 5 |
| Time, | permeation | permeation | 25 μM | 50 μM | 100 μM |
| mins | enhancers | enhancers | PN159 | PN159 | PN159 |
| 0 | 183.825 | 257.3 | 228.675 | 424.4 | 294.225 |
| 2.5 | 1280.7 | 242.8 | 526.375 | 749.975 | 1748.225 |
| 5 | 1449.425 | 273.675 | 1430.15 | 1293.4 | 3088.2 |
| 10 | 8251.8 | 372.05 | 6521.7 | 12517.2 | 14486.6 |
| 15 | 13731.2 | 398.225 | 12563.075 | 34455.3 | 20882.725 |
| 30 | 19537.55 | 476.475 | 15222.6 | 35294.375 | 25470.475 |
| 45 | 13036.075 | 340.7 | 9081.125 | 21582.225 | 16499.55 |
| 60 | 7080.875 | 283.825 | 4843.15 | 9461.925 | 10676.625 |
| 120 | 1671.9 | 192.575 | 1224.2 | 2337.775 | 1891.275 |

The pharmacokinetic data calculated from the above data is shown below in Table 8:

TABLE 8
Summary of Pharmacokinetic Data

| Variable | Group | Mean | SD | SE |
|---|---|---|---|---|
| Cmax (pg/mL) | 1 | 19832.18 | 17737.21 | 8868.605 |
| Tmax (min) | 1 | 32.5 | 20.6155 | 10.3078 |
| AUClast | 1 | 991732.1 | 930296.3 | 465148.1 |
| (min*pg/mL) | | | | |
| AUCINF | 1 | 1357132 | 928368.5 | 535993.8 |
| (min*pg/mL) | | | | |
| t½ (min) | 1 | 23.69 | 1.713 | 0.989 |
| Cmax (pg/mL) | 2 | 516.725 | 196.492 | 98.246 |
| Tmax (min) | 2 | 26.25 | 14.3614 | 7.1807 |
| AUClast | 2 | 36475.72 | 9926.104 | 4963.052 |
| (min*pg/mL) | | | | |
| AUCINF | 2 | 60847.41 | 17688.31 | 8844.156 |
| (min*pg/mL) | | | | |
| t½ (min) | 2 | 84.5919 | 26.8859 | 13.4429 |
| Cmax (pg/mL) | 3 | 15533.95 | 13225.88 | 6612.941 |
| Tmax (min) | 3 | 22.5 | 8.6603 | 4.3301 |
| AUClast | 3 | 748104.1 | 661213.8 | 330606.9 |
| (min*pg/mL) | | | | |
| AUCINF | 3 | 796354.7 | 721017.8 | 360508.9 |
| (min*pg/mL) | | | | |
| t½ (min) | 3 | 24.8467 | 4.3108 | 2.1554 |
| Cmax (pg/mL) | 4 | 40995.53 | 32112.71 | 16056.35 |
| Tmax (min) | 4 | 26.25 | 7.5 | 3.75 |
| AUClast | 4 | 1692499 | 1339896 | 669947.8 |
| (min*pg/mL) | | | | |
| AUCINF | 4 | 1787348 | 1395185 | 697592.4 |
| (min*pg/mL) | | | | |
| t½ (min) | 4 | 25.5355 | 8.6139 | 4.3069 |
| Cmax (pg/mL) | 5 | 27974.4 | 17584.31 | 8792.154 |
| Tmax (min) | 5 | 33.75 | 18.8746 | 9.4373 |
| AUClast | 5 | 1384241 | 817758.8 | 408879.4 |
| (min*pg/mL) | | | | |
| AUCINF | 5 | 1518949 | 1030623 | 595030.3 |
| (min*pg/mL) | | | | |
| t½ (min) | 5 | 20.4628 | 6.5069 | 3.7568 |

Compared with the Group 2 (no enhancer) formulation, the following relative enhancement ratios were determined (Table 9):

TABLE 9
Relative Enhancement Ratios

| | | Relative | Relative |
|---|---|---|---|
| Group | Formulation | Cmax | AUC last |
| 1 | Small molecule permeation enhancers | 38× | 27× |
| 3 | PN159, 25 μm | 30× | 21× |
| 4 | PN159, 50 μm | 79× | 46× |
| 5 | PN159, 100 μm | 54× | 38× |

The foregoing data demonstrate that TJMP enhances in vivo intranasal permeation of a human hormone peptide therapeutic to an equal or greater degree compared to small molecule permeation enhancers. The greatest effect of the peptide is seen at a 50 μM concentration. The 100 μM concentration resulted in somewhat less permeation, although both resulted in higher permeation than the small molecule permeation enhancers.

### Example 6

### Permeation Enhancement by TJMP for an Oligopeptide Therapeutic Agent

The present example demonstrates efficacy of an exemplary peptide of the invention, PN159 to enhance epithelial permeation for a cyclic pentapeptide, melanocortin-4 receptor agonist (MC-4RA) a model oligopeptide agonist for a mammalian cellular receptor. In this example, a combination of one or more of the permeabilizing peptides with MC-4RA is described. Useful formulations in this context can include a combination of an oligopeptide therapeutic, a permeabilizing peptide, and one or more other permeation enhancers. The formulation may also contain buffers, tonicifying agents, pH adjustment agents, and peptide/protein stabilizers such as amino acids, sugars or polyols, polymers, and salts.

The effect of PN159 on permeation of MC-4RA was evaluated in this study. MC-4RA was a methanesulphonate salt with a molecular weight of approximately 1,100 Da, which modulates activity of the MC-4 receptor. The PN159 concentrations evaluated are 5, 25, 50, and 100 μM. 45 mg/ml M-β-CD was used as a solubilizer for all formulations to achieve 10 mg/ml peptide concentration. The effect of PN159 was assessed either by itself or in combination with EDTA (1, 2.5, 5, or 10 mg/ml). The formulation pH was fixed at 4 and the osmolarity was at 220 mOsm/kg.

HPLC Method

The concentrations of MC-4RA in the basolateral media was analyzed by the RP-HPLC using a C18 RP chromatography with a flow rate of 1 mL/minute and a column temperature of 25° C.
Solvent A: 0.1% TFA in water; Solvent B: 0.1% TFA in ACN
Injection Volume: 50 μL
Detection: 220 nm
Run Time: 15 min

MC-4RA was combined with 5, 25, 50, and 100 μM PN159, pH 4 and osmolarity ˜220 mOsm/kg. The combination was tested using an in vitro epithelial tissue model to monitor PTH permeation, transepithelial electrical resistance (TER), and the cytotoxicity of the formulation by MTT and LDH assays.

The results of studies of the permeation of MC-4RA evinced that TJMP, in addition to enhancing mucosal permeation for peptide hormone therapeutics, significantly enhanced epithelial permeation for an oligopeptide therapeutic agent.

### Example 7

### Permeation Enhancement by TJMP for a Small Molecule Drug

The present example demonstrates efficacy of an exemplary peptide of the invention, PN159, to enhance epithelial permeation for a small molecule drug, exemplified by the acetylcholinesterase (ACE) inhibitor galantamine. In this example, a combination of one or more of the permeabilizing peptides with a small molecule drug is described. Useful formulations in this context can include a combination of a small molecule drug, a permeabilizing peptide, and one or more other permeation enhancers. The formulation may also contain buffers, tonicifying agents, pH adjustment agents, stabilizers and/or preservatives.

The present invention combines galantamine with PN159 to enhance permeation of galantamine across the nasal mucosa. This increase in drug permeation is unexpected because galantamine is a small molecule that can permeate the nasal epithial membrane independently. The significant enhancement of galantamine permeation across epithelia mediated by addition of excipients which enhance the permeation of peptides is therefore surprising, on the basis that such excipients would not ordinarily be expected to significantly increase permeation of galantamine across the epithelial tissue layer. The invention therefore will facilitate nasal delivery of galantamine and other small molecule drugs by increasing their bioavailability.

In the present studies, 40 mg/ml galantamine in the lactate salt form was combined with 25, 50, and 100 μM PN159 in solution, pH 5.0 and osmolarity ˜270 mOsm. The combination was tested using an in vitro epithial tissue model to monitor galantamine permeation, transepithelial electrical resistance (TER), and the cytotoxicity of the formulation by LDH and MTT assays as described above. Permeation measurements for galantamine were conducted by standard HPLC analysis, as follows.

HPLC Analysis

Galantamine concentration in the formulation and in the basolateral media (permeation samples) was determined using an isocratic LC (Waters Alliance) method with UV detection.
Column: Waters Symmetry Shield, C18, 5 um, 25×0.46 cm
Mobile phase: 5% ACN in 50 mM ammonium formate, pH 3.0
Flow rate: 1 ml/min
Column temperature: 30° C.
Calibration curve: 0-400 μg/ml Galantamine HBr
Detection: UV at 285 nm

Based on the foregoing studies, PN159 improves transmucosal delivery of small molecules. Galantamine was chosen as a model low molecular weight drug, and the results for this molecule are considered predictive of permeabilizing peptide activity for other small molecule drugs. To evaluate permeabilizing activity in this context, 40 mg/ml galantamine in the lactate salt form was combined with 25, 50, and 100 μM PN159 in solution, pH 5.0 and osmolarity ˜270 mOsm. The combination was tested using an in vitro epithelal tissue model to monitor galantamine permeation, transepithelial electrical resistance (TER), and the cytotoxicity of the formulation by LDH and MTT assays.

In the in vitro tissue model, the addition of PN159 resulted in a dramatic increase in drug permeation across the cell barrier. Specifically, there was a 2.5-3.5 fold increase in the Pₐₚₚ of 40 mg/ml galantamine.

PN159 reduced TER in the presence of galantamine just as described in previous examples.

Cell viability remained high (>80%) in the presence of galantamine lactate and PN159 at all concentrations tested. Conversely, cyclotoxicity was low in the presence of PN159 and galantamine lactate, as measured by LDH. Both of these assays suggest that PN159 is not toxic to the epithelial membrane.

In the absence of PN159, the Pₐₚₚ for galantamine was about 2.1×10⁻⁶ cm/s. In the presence of 25, 50 and 100 mM PN159, Pₐₚₚ was 5.1×10⁻⁶, 6.2×10⁻⁶, and 7.2×10⁻⁶ cm/s, respectively. Thus, the PN159 afforded a 2.4- to 3.4-fold increase in Pₐₚₚ of this model low-molecular-weight drug.

TJMP surprisingly increased epithelial permeation of galantamine as a model low molecular weight drug. The addition of PN159 to galantamine in solution significantly enhanced galantamine permeation across epithelial monolayers. Evidence shows that PN159 temporarily reduced TER across the epithelial membrane without damaging the cells in the membrane, as measured by high cell viability and low cytotoxicity. TJMP enhanced bioavailability of galantamine and other small molecule drugs in vivo via the same mechanism that is demonstrated herein using in vitro models. It is further expected that TJMP will enhance permeation of galantamine at higher concentrations as well.

### Example 8

### Permeation Enhancement by TJMP for Proteins

Having established the utility of the PN159 for transmucosal formulations of low-molecular-weight compounds, it was important to discern whether these observations could be extrapolated to larger molecules, e.g., therapeutic peptides and proteins. For this purpose, in vitro tissue studies were performed on salmon calcitonin as a model therapeutic peptide in the absence and presence of 25, 50, and 100 mM PN159. In the absence of PN159, the Pₐₚₚ for calcitonin was about 1×10⁻⁷ cm/s, about an order of magnitude lower than that for galantamine, presumably due to the difference in molecular weight. The data reveal a dramatic increase in calcitonin permeation in the presence of the PN159, up to a 23- to 47-fold increase in Pₐₚₚ compared to the case of the calcitonin alone (Table 10).

TABLE 10
Pₐₚₚ Measured Using the in vitro Tissue Model

| | [PN159] | Pₐₚₚ | |
|---|---|---|---|
| Drug Formulation | (μM) | (cm/s) | Relative Pₐₚₚ |
| Galantamine | 0 | 2.1 × 10⁻⁶ | 1.0 |
| 40 mg/mL, pH 5.0 | 25 | 5.1 × 10⁻⁶ | 2.4 |
| | 50 | 6.2 × 10⁻⁶ | 3.0 |
| | 100 | 7.2 × 10⁻⁶ | 3.4 |
| Calcitonin | 0 | 9.7 × 10⁻⁸ | 1.0 |
| 1 mg/mL, pH 3.5 | 25 | 2.2 × 10⁻⁶ | 23. |
| | 50 | 3.3 × 10⁻⁶ | 34. |
| | 100 | 4.6 × 10⁻⁶ | 47. |
| PTH₁₋₃₄ | 0 | 1.1 × 10⁻⁷ | 1.0 |
| 1 mg/mL, pH 4.5 | 25 | 3.4 × 10⁻⁷ | 3.0 |
| | 50 | 4.9 × 10⁻⁷ | 4.5 |
| | 100 | 4.3 × 10⁻⁷ | 3.9 |
| PYY₃₋₃₆ | 0^{a} | 1.3 × 10⁻⁷ | 1.0 |
| 1 mg/mL, pH 7.0 | 25 | 1.6 × 10⁻⁶ | 12. |
| | 100 | 2.2 × 10⁻⁶ | 17. |

^{a}pH was 5.0

In order to explore the generality of these findings, two additional peptides, namely human parathyroid hormone 1-34 (PTH₁₋₃₄) and human peptide YY 3-36 (PYY₃₋₃₆) were examined in the in vitro model in the absence and presence of PN159 (Pₐₚₚ data presented in Table 10). In the absence of PN159, the Pₐₚₚ of these two peptides was consistent to that for calcitonin. In the case of PTH₁₋₃₄, the presence of PN159 afforded about 3-5 fold increase in Pₐₚₚ. When PYY₃₋₃₆ was formulated in the presence of PN159, the Papp was increased about 12- to 17-fold. These data confirm the generality of our finding that the TJMP enhanced transmucosal drug delivery for small molecules and proteins.

### Example 9

### Chemical Stability of TJMP

The chemical stability of the PN159 was determined under therapeutically relevant storage conditions. A stability indicating HPLC method was employed. Solutions (50 mM) were stored at various pH (4.0, 7.3, and 9.0) and temperature (5° C., 25° C., 35° C., 40° C., and 50° C.) conditions. Samples at pH 4 contained 10 mM citrate buffer. Samples at pH 7.3 and 9.0 contained 10 mM phosphate buffer. Storage stability results (including the Arrhenius plot) show that PN159 was most chemically stable at low temperature and pH. For example, at 5° C. and pH 4.0 or pH 7.3, there was essentially 100% recovery of PN159 for six month storage. When the storage temperature was increased to 25° C., there was a 7% and 26% loss of native PN159 for samples at pH 4 or pH 7, respectively, after six months. At pH 9 and/or at elevated temperature, e.g., 40 to 50° C., rapid deterioration of the PN159 ensued. The pH range of 4.0 to 7.3 and the temperature range of refrigerated to ambient are most relevant for intranasal formulations. Therefore, these data support that the TJMP can maintain chemical integrity under storage conditions relevant to IN formulations.

Although the foregoing invention has been described in detail by way of example for purposes of clarity of understanding, it will be apparent to the artisan that certain changes and modifications may be practiced within the scope of the appended claims which are presented by way of illustration not limitation. In this context, various publications and other references have been cited within the foregoing disclosure for economy of description. Each of these references is incorporated herein by reference in its entirety for all purposes. It is noted, however, that the various publications discussed herein are incorporated solely for their disclosure prior to the filing date of the present application, and the inventors reserve the right to antedate such disclosure by virtue of prior invention.

### Example 10

### In Vivo Evaluation of Tight Junction Modulating Peptides in Rabbits by Intranasal Administration

A pharmacokinetic (PK) study in rabbits was performed to evaluate the plasma pharmacokinetic properties of Peptide YY (PYY) with various tight junction modulating peptides (TJMPs) administered via intranasal (IN) delivery.

Animal Model

In this study, New Zealand White rabbits (Hra: (NZW) SPF) were used as test subjects to evaluate plasma pharmacokinetics of MC-4RA by intranasal administration and intravenous infusion. The treatment of animals was in accordance with regulations outlined in the USDA Animal Welfare Act (9 C.F.R., Parts 1, 2, and 3) and the conditions specified in the Guide for the Care and Use of Laboratory Animals, ILAR publication, 1996, National Academy Press).

Rabbits were chosen as animal subjects for this study because the pharmacokinetic profile derived from a drug administered to rabbits closely resembles the PK profile for the same drug in humans.

Dose Administration

The experimental design and dosing regime for the 9 TJMPs tested is summarized in Table 11. All experimental groups were given 205 μg/kg PYY(3-36) in combination with an individual TJMP or phosphate buffered saline (PBS; negative control) by intranasal (IN) administration. Each formulation was administered once into the left nares using a pipetteman and disposable plastic tip. The head of the animal was tilted back and the dose was administered at the time of inhalation by the animal so as to allow capillary action to draw the solution into the nares. Following IN administration, the animal's head was restrained in the tilted back position for about 15 seconds to prevent any loss of the administered dose. During the procedure, extreme care was taken to avoid any tissue damage potentially resulting from contact with intranasal mucosa.

TABLE 11
Summary of Test Groups

| | Number of | | Tight Junction Modulator | PYY3 |
|---|---|---|---|---|
| Group | Animals | Route | (Concentration) | (ug/kg) |
| 1 | 5M | Intranasal | PBS | 205 |
| 2 | 5M | Intranasal | PN159 (50 μM) | 205 |
| 3 | 5M | Intranasal | PN161 (100 μM) | 205 |
| 4 | 5M | Intranasal | PN202 (100 μM) | 205 |
| 5 | 5M | Intranasal | PN27 (250 μM) | 205 |
| 6 | 5M | Intranasal | PN58 (500 μM) | 205 |
| 7 | 5M | Intranasal | PN73 (500 μM) | 205 |
| 8 | 5M | Intranasal | PN228 (500 μM) | 205 |
| 9 | 5M | Intranasal | PN183 (1000 μM) | 205 |
| 10 | 5M | Intranasal | PN556 (1000 μM) | 205 |

PN556 has the same primary sequence as PN283, but has no maleimide modification at the N-terminus of the peptide.

Blood and Plasma Sample Collection

Following does administration by IN, serial blood samples were taken from each animal by direct venipuncture of a marginal ear vein. Blood samples were collected at predose, 5, 10, 15, 20, 30, 45, 60, 90, 120 and 180 minutes post-dosing. Samples were collected in tubes containing dipotassium EDTA as the anticoagulant. The tubes were chilled until configuration. All samples were centrifuged within 1 hour of collection. Plasma was harvested and transferred into prelabled plastic vials, frozen in a dry ice/acetone bath, and then stored at approximately −70° C. until a pharmacokinetic analysis was performed.

Clinical observations were made at each blood sampling time and an examination of both nostrils for all animals in the IN administration test groups was conducted just prior to 5 minutes and 1 hour post-intranasal dosing.

Analytical Method

Samples from each animal in all study groups were analyzed for PYY (3-36) levels using by ELISA. The test articles prior to and after dosing were run on HPLC for quality control. Aliquots (0.1 mL) of plasma were protein precipitated with acetonitrile after adding a bio-analytical internal standard. The supernatant was dried with nitrogen, reconstituted in HPLC buffer and then injected onto a HPLC system. The effluent is detected by positive ion electrospray ionization tandem triple quadrupole mass spectrometer. The PK data was analyzed by WinNonlin (Pharsight Corp., Mountain View).

Results

The mean plasma PK parameters for each test group are summarized in Table 12. No adverse clinical signs were observed following administration of any formulations. Post-intranasal examination of both nostrils of animals administered formulations via IN revealed neither any redness, nor swelling. The PK study evaluated the Cₘₐₓ (maximum observed concentration), Tₘₐₓ (time of maximum concentration) and AUC (Area Under the Curve) last and infinity (inf). Eight TJMPs were ranked and categorized into 4 different performance tiers according to their level of in vivo permeability with Tier I containing TJMPs with the greatest level of in vivo permeability and each subsequent Tier containing TJMPs with progressively decreasing levels of in vivo permeability.

TABLE 12
Summary of Pharmacokinetic Data

| | In Vivo | | | | | |
|---|---|---|---|---|---|---|
| | Tier | | Tₘₐₓ | Cₘₐₓ | AUClast | AUCinf |
| Group | Ranking | T_{1/2} | (min) | (pg/mL) | (min*pg/mL) | (min*pg/mL) |
| PBS | | 86.0 | 22.0 | 806 | 4.5 × 10⁴ | 6.81 × 10⁴ |
| PN159 | I | 30.2 | 17.0a | 30200 | 1.52 × 10⁶ | 1.55 × 10⁶ |
| PN161 | I | 34.3 | 24.0 | 32100 | 1.62 × 10⁶ | 1.65 × 10⁶ |
| PN27 | I | 29.9 | 33.0 | 29300 | 1.67 × 10⁶ | 1.71 × 10⁶ |
| PN228 | II | 30.4 | 31.0 | 21200 | 1.06 × 10⁶ | 1.08 × 10⁶ |
| PN202 | II | 34.1 | 32.0 | 12700 | 7.35 × 10⁵ | 7.63 × 10⁵ |
| PN58 | III | 29.5 | 43.0 | 12800 | 8.3 × 10⁵ | 8.71 × 10⁵ |
| PN73 | IV | 53.8 | 37.0 | 8220 | 3.46 × 10⁵ | 3.55 × 10⁵ |
| PN183 | IV | 33.7 | 22.0 | 5450 | 2.58 × 10⁵ | 2.75 × 10⁵ |
| PN556 | IV | 51.2 | 22.0 | 4620 | 2.47 × 10⁵ | 2.80 × 10⁵ |

These data show that the in vivo permeability observed for both PN161 and PN27 is comparable to PN159; and the remaining TJMPs, at the concentrations tested, achieved a level of in vivo permeability below that of PN159.

What is claimed is:

1. A pharmaceutical formulation comprising a biologically active agent and a mucosal delivery-enhancing effective amount of a permeabilizing peptide that reversibly enhances mucosal epithelial transport of the biologically active agent in a mammalian subject, wherein the permeabilizing peptide is a tight junction modulating peptide (TJMP), a TJMP analogue, a conjugate of a TJMP, a conjugate of a TJMP analogue, or a complex thereof.

2. The formulation of claim 1, wherein the TJMP is selected from the group consisting of

| | |
|---|---|
| CNGRCGGKKKLKLLLKLL | (SEQ ID NO: 32) |
| LRKLRKRLLRLRKLRKRLLR-amide | (SEQ ID NO: 33) |

3. The composition of claim 2, wherein said TJMP is conjugated to at least one water soluble chain.

4. The formulation of claim 3, wherein the water soluble chain is a poly(alkylene oxide) chain.

5. The formulation of claim 4, wherein such poly(alkylene oxide) chain is a polyethylene glycol (PEG) chain.

6. The formulation of claim 5, wherein the PEG has a molecular size between about 0.2 and about 200 kiloDaltons (kDa).

7. The formulation of claim 1, wherein the TJMP decreases electrical resistance across a mucosal tissue barrier.

8. The formulation of claim 7, where the decrease in electrical resistance is at least 80% of the electrical resistance prior to applying the TJMP.

9. The formulation of claim 1, wherein the TJMP increases permeability of the molecule across a mucosal tissue barrier.

10. The formulation of claim 9 wherein the increase in permeability is at least two fold.

11. The formulation of claim 9, wherein the permeability is paracellular.

12. The formulation of claim 9, wherein the increased permeability results from modification of tight junctions.

13. The formulation of claim 9, wherein the permeability is transcellular, or a combination of trans- and paracellular.

14. The formulation of claims 9, wherein the mucosal tissue layer is comprised of an epithelial cell layer.

15. The formulation of claim 14, wherein the epithelial cell is selected from the group consisting of tracheal, bronchial, alveolar, nasal, pulmonary, gastrointestinal, epidermal or buccal.

16. The formulation of claim 15, wherein the epithelial cell is nasal.

17. The formulation of claim 1, wherein the biologically active agent is a peptide or protein.

18. The formulation of claim 17, wherein the peptide or protein is comprised of between 2 and 1000 amino acids.

19. The formulation of claim 17, wherein the peptide or protein is comprised of between 2 and 50 amino acids.

20. The formulation of claim 17, wherein the peptide or protein is cyclic.

21. The formulation of claim 17, wherein the peptide or protein forms dimers or higher-order oligomers via physical or chemical bonding.

22. The formulation of claim 17, wherein the peptide or protein is selected from the group comprising GLP-1, PYY₃₋₃₆, PTH₁₋₃₄ and Exendin-4.

23. The formulation of claim 17, wherein the biologically active agent is a protein.

24. The formulation of claim 23, wherein the protein is selected from the group consisting of beta-interferon, alpha-interferon, insulin, erythropoietin, G-CSF, and GM-CSF, growth hormone, and analogues of any of these.

25. A method of administering a molecule to an animal comprising preparing a formulation as in claim 1 and bringing such formulation in contact with a mucosal surface of such animal.

26. The method of claim 25, wherein such mucosal surface is intranasal.

27. A dosage form comprising the formulation of claim 1, wherein the dosage form is liquid.

28. The dosage form of claim 27, wherein the liquid is in the form of droplets.

29. A dosage form comprising the formulation of claim 1, wherein the dosage form is solid.

30. The dosage form of claim 29, wherein solid is reconstituted in liquid prior to administration.

31. The dosage form of claim 29, wherein the solid is administered as a powder.

32. The dosage form of claim 29, wherein the solid is in the form of a capsule, tablet or gel.

33. A molecule that reversibly enhances mucosal epithelial transport of a biologically active agent in a mammalian subject, comprising a tight junction modulating peptide (TJMP) or a TJMP analogue.

34. The molecule of claim 33, wherein the TJMP is selected from the group consisting of

| | |
|---|---|
| CNGRCGGKKKLKLLLKLL | (SEQ ID NO: 32) |
| LRKLRKRLLRLRKLRKRLLR-amide | (SEQ ID NO: 33) |

35. The molecule of claim 34, wherein the TJMP is covalently linked to a poly(alkylene oxide) chain.

36. The molecule of claim 35, wherein such poly(alkylene oxide) chain is a polyethylene glycol (PEG) chain.

37. The molecule of claim 36, wherein the PEG has a molecular size between about 0.2 and about 200 kiloDaltons (kDa).
